(19) United States
(12) Patent Application Publication
(10) Pub. No.: US 2013/0012591 A1
(43) Pub. Date: Jan. 10, 2013

(54) PROPHYLAXIS OF SKIN CANCER WITH RETINAMIDES

(75) Inventors: Barry Scott Butler, Tampa, FL (US); Roger Vogel, Sarasota, FL (US); Nathan L. Mata, San Diego, CA (US); Caryn L. Peterson, Encinitas, CA (US)

(73) Assignee: Revision Therapeutics, Inc., San Diego, CA (US)

(21) Appl. No.: 13/504,467

(22) PCT Filed: Oct. 28, 2010
(86) PCT No.: PCT/US10/54575

§ 371 Date: Sep. 28, 2012

Related U.S. Application Data
(60) Provisional application No. 61/255,739, filed on Oct. 28, 2009.
(60) Provisional application No. 61/372,821, filed on Aug. 11, 2010.

(51) Int. Cl. A61K 31/167; A61P 35/00; C07C 235/30

(52) U.S. Cl. 514/613; 564/123

(57) ABSTRACT

Provided in certain embodiments herein are methods of prophylaxis of skin cancer in individuals having a heightened risk of skin cancer with a fenretinide agent.

### CROSS-REFERENCE

This application claims priority from U.S. Provisional Application 61/255,739, filed 28 Oct., 2009, and U.S. Provisional Application 61/372,821, filed 11 Aug., 2010; both of which are incorporated herein in their entirety.

### BACKGROUND OF THE INVENTION

Skin cancer is a malignant growth on the skin. About one million people are diagnosed with skin cancer in the United States every year. There are about twelve thousand deaths from skin cancer every year in the United States.

### SUMMARY OF THE INVENTION

Disclosed herein, in certain embodiments, is a method of treating or reducing recurrence of a non-melanoma skin cancer in an individual diagnosed with excessive lipofuscin accumulation, a macular dystrophy, Stargardt's disease, GA, non-exudative AMD, and/or exudative AMD comprising administering to the individual an effective amount of an active agent that (a) decreases serum retinol; (b) increases ceramide levels; (c) decreases the activity of or blocks a sigma receptor; and/or (d) decreases the activity of or blocks the patched or smoothened receptor within the hedgehog pathway. In some embodiments, the active agent is a retinoid or a retinoid derivative. In some embodiments, the active agent is N-(4-hydroxyphenyl) retinamide, N-(4-methoxyphenyl)retinamide, 4-oxo-N-(4-hydroxyphenyl)retinamide, a compound of Formula (I):

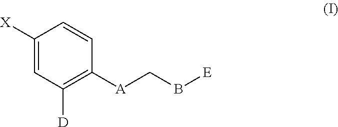

wherein:

### A is O, NH, or S;

B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
D is isopropyl, isobutyl, sec-butyl, tert-butyl, neopentyl, sec-pentyl, isopentyl, cyclopropyl, cyclobutyl, cyclopentyl, methylenecyclopropyl, methylenecyclobutyl, methylenecyclopentyl;
E is (C═O)—OR, —O—(C═O)—R, —(C═O)—R, —OR, a carboxylic acid bioisostere, —(C═O)—NR¹R,
NR¹—(C═O)—R, —(C₁-C₇)alkyl-(C═O)—OR, or —(C₁-C₇)alkyl-(C═O)—NR¹R;

### R is H or

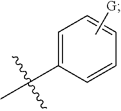

G is —OR¹, —(C₁-C₆)alkyl, —(C₁-C₆)alkyl-OR¹, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
X is a halogen;
or an active metabolite, or a pharmaceutically acceptable prodrug, salt, or solvate thereof;
or a combination thereof. In some embodiments, the individual has elevated plasma retinol; elevated plasma RBP4; elevated concentrations of sigma receptors, optionally in an eye; or elevated concentrations of VEGF, optionally in an eye or a cancerous tumor; or a apo-RBP-to-holo-RBP ratio above 0.5. In some embodiments, the individual is (a) a male human individual having plasma RBP4 concentration that is greater than 25 μg/mL and/or a plasma apo-RBP-to-holo-RBP ratio above 0.5; or (b) a female human individual having plasma RBP4 concentration that is greater than 20 μg/mL and/or a plasma apo-RBP-to-holo-RBP ratio above 0.5. In some embodiments, the effective amount of the active agent is an amount sufficient to reduce the level of a risk factor associated with AMD or a non-melanoma skin cancer in the individual by about 25% to about 75%; wherein the risk factor is selected from: elevated concentrations of circulating vitamin A, elevated concentrations of circulating RBP, elevated concentrations of circulating holo-RBP, elevated concentrations of VEGF, or elevated concentrations of sigma receptors. In some embodiments, the effective amount of the active agent is less than about 300 mg daily. In some embodiments, the effective amount of the active agent is about 50 mg to about 150 mg daily. In some embodiments, the skin cancer is a non-melanoma skin cancer. In some embodiments, the skin cancer is basal cell carcinoma or squamous cell carcinoma.

Disclosed herein, in certain embodiments, is a use of a retinoid or a retinoid derivative for the manufacture of a medicament for the treatment of non-melanoma skin cancer in an individual diagnosed with excessive lipofuscin accumulation, a macular dystrophy, Stargardt's disease, GA, non-exudative AMD, and/or exudative AMD. In some embodiments, the retinoid or retinoid derivative is N-(4-hydroxyphenyl) retinamide, N-(4 methoxyphenyl)retinamide, 4-oxo-N-(4-hydroxyphenyl)retinamide, a compound of Formula (I):

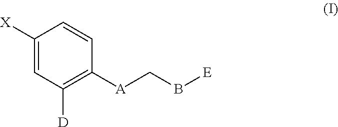

wherein:

### A is O, NH, or S;

B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
D is isopropyl, isobutyl, sec-butyl, tert-butyl, neopentyl, sec-pentyl, isopentyl, cyclopropyl, cyclobutyl, cyclopentyl, methylenecyclopropyl, methylenecyclobutyl, methylenecyclopentyl;
E is (C═O)—OR, —O—(C═O)—R, —(C═O)—R, —OR, a carboxylic acid bioisostere, —(C═O)—NR¹R, NR—(C═O)—R, —(C₁-C₇)alkyl-(C═O)—OR, or —(C₁-C₇)alkyl-(C═O)—NR¹R;

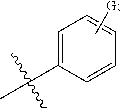

### R is H or

G is —OR¹, —(C₁-C₆)alkyl, —(C₁-C₆)alkyl-OR¹, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
X is a halogen;
or an active metabolite, or a pharmaceutically acceptable prodrug, salt, or solvate thereof;
or a combination thereof.

Disclosed herein, in certain embodiments, is a method of treating Gorlin's Syndrome, comprising administering to the individual an effective amount of an active agent that (a) decreases serum retinol; (b) increases ceramide levels; (c) decreases the activity of or blocks a sigma receptor; and/or (d) decreases the activity of or blocks the patched or smoothened receptor within the hedgehog pathway. In some embodiments, the active agent is a retinoid or a retinoid derivative. In some embodiments, the active agent is N-(4-hydroxyphenyl) retinamide, N-(4-methoxyphenyl)retinamide, 4-oxo-N-(4-hydroxyphenyl)retinamide, a compound of Formula (I):

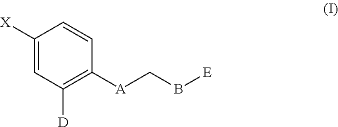

wherein:

### A is O, NH, or S;

B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
D is isopropyl, isobutyl, sec-butyl, tert-butyl, neopentyl, sec-pentyl, isopentyl, cyclopropyl, cyclobutyl, cyclopentyl, methylenecyclopropyl, methylenecyclobutyl, methylenecyclopentyl;
E is (C═O)—OR, —O—(C═O)—R, —(C═O)—R, —OR, a carboxylic acid bioisostere, —(C═O)—NR¹R, NR¹—(C═O)—R, —(C₁-C₇)alkyl-(C═O)—OR, or —(C₁-C₇)alkyl-(C═O)—NR¹R;

### R is H or

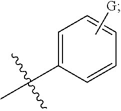

G is —OR¹, —(C₁-C₆)alkyl, —(C₁-C₆)alkyl-OR¹, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
X is a halogen;
or an active metabolite, or a pharmaceutically acceptable prodrug, salt, or solvate thereof;
or a combination thereof. In some embodiments, the method further comprises treating or reducing the recurrence of a basal cell carcinoma.

Disclosed herein, in certain embodiments, is a use of a retinoid or a retinoid derivative for the manufacture of a medicament for the treatment of Gorlin's Syndrome. In some embodiments, the retinoid or retinoid derivative is N-(4-hydroxyphenyl) retinamide, N-(4-methoxyphenyl)retinamide, 4-oxo-N-(4-hydroxyphenyl)retinamide, a compound of Formula (I):

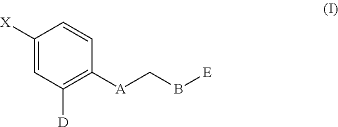

wherein:

### A is O, NH, or S;

B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
D is isopropyl, isobutyl, sec-butyl, tert-butyl, neopentyl, sec-pentyl, isopentyl, cyclopropyl, cyclobutyl, cyclopentyl, methylenecyclopropyl, methylenecyclobutyl, methylenecyclopentyl;
E is (C═O)—OR, —O—(C═O)—R, —(C═O)—R, —OR, a carboxylic acid bioisostere, —(C═O)—NR¹R, NR—(C═O)—R, —(C₁-C₇)alkyl-(C═O)—OR, or —(C₁-C₇)alkyl-(C═O)—NR¹R;

### R is H or

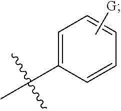

G is —OR¹, —(C₁-C₆)alkyl, —(C₁-C₆)alkyl-OR¹, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
X is a halogen;
or an active metabolite, or a pharmaceutically acceptable prodrug, salt, or solvate thereof;
or a combination thereof.

Described herein, in certain embodiments, are methods for treating certain types of cancer in patients that have macular or retinal degenerations comprising administering an agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments the cancer is a skin cancer and/or basal cell carcinoma.

Provided herein, in some embodiments, are methods of reducing the incidences of cancer (e.g., skin cancer or conjunctival malignant melanoma) in an individual in need thereof comprising administering to the individual an effective amount of a therapeutic agent, the therapeutic agent being N-(4-hydroxyphenyl)retinamide (4-HPR), N-(4-methoxyphenyl)retinamide (4-MPR), 4-oxo-N-(4-hydroxyphenyl)retinamide (4-oxo-4-HPR), a compound of Formula (I), or a combination thereof. Provided herein, in certain embodiments, are methods of reducing the incidences of skin cancer in an individual in need thereof comprising administering to the individual an effective amount of a therapeutic agent, the therapeutic agent being N-(4-hydroxyphenyl)retinamide (4-HPR), N-(4 methoxyphenyl)retinamide (4-MPR), 4-oxo-N-(4-hydroxyphenyl)retinamide (4-oxo-4-HPR), a compound of Formula (I), or a combination thereof. Provided herein, in some embodiments, are methods of reducing the incidences of skin cancer in an individual diagnosed with geographic atrophy (GA) or age-related macular degeneration (AMD) comprising administering to the individual an effective amount of a therapeutic agent, the therapeutic agent being N-(4-hydroxyphenyl) retinamide, N-(4-methoxyphenyl)retinamide, 4-oxo-N-(4-hydroxyphenyl)retinamide, a compound of Formula (I) or a combination thereof. Further disclosed herein, in some embodiments, are methods of prophylactically treating skin cancer comprising administering to an individual in need thereof an effective amount of a therapeutic agent, the therapeutic agent being N-(4-hydroxyphenyl)retinamide, N-(4-methoxyphenyl)retinamide, 4-oxo-N-(4-hydroxyphenyl)retinamide, a compound of Formula (I) or a combination thereof. Provided herein, in certain embodiments, are methods of prophylactically treating basal cell carcinoma, squamous cell carcinoma, or melanoma in an individual in need thereof comprising administering to the individual an effective amount of a therapeutic agent, the therapeutic agent being N-(4-hydroxyphenyl)retinamide, N-(4-methoxyphenyl)retinamide, 4-oxo-N-(4-hydroxyphenyl)retinamide, a compound of Formula (I) or a combination thereof. Compounds of Formula (I) include compounds having the following structure:

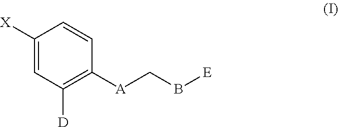

wherein:
A is O, NH, or S;
B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
D is isopropyl, isobutyl, sec-butyl, tert-butyl, neopentyl, sec-pentyl, isopentyl, cyclopropyl, cyclobutyl, cyclopentyl, methylenecyclopropyl, methylenecyclobutyl, methylenecyclopentyl;
E is (C═O)—OR, —O—(C═O)—R, —(C═O)—R, —OR, a carboxylic acid bioisostere, —(C═O)—NR¹R, NR¹—(C═O)—R, —(C₁-C₇)alkyl-(C═O)—OR, or —(C₁-C₇)alkyl-(C═O)—NR¹R;
R is H or

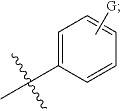

G is —OR¹, —(C₁-C₆)alkyl, —(C₁-C₆)alkyl-OR¹, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
X is a halogen;
   or an active metabolite, or a pharmaceutically acceptable prodrug, salt, or solvate thereof. In some embodiments, compounds of Formula (I) include compounds of Formula (II):

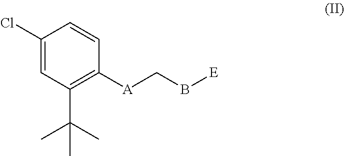

wherein:
A is O, NH, or S;
B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
E is (C═O)—OR, —O—(C═O)—R, —(C═O)—R, —OR, a carboxylic acid bioisostere, —(C═O)—NR¹R, NR¹—(C═O)—R, —(C₁-C₇)alkyl-(C═O)—OR, or —(C₁-C₇)alkyl-(C═O)—NR¹R;
R is H or

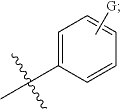

G is —OR¹, —(C₁-C₆)alkyl, —(C₁-C₆)alkyl-OR¹, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
   or an active metabolite, or a pharmaceutically acceptable prodrug, salt, or solvate thereof. In specific embodiments, a compound of Formula (I) or (II) is a compound wherein A is O. In a further embodiment, a compound of Formula (I) or (II) is a compound wherein B is —(CH₂)ₙ and n is 1-6, or B is —(C₃-C₈)cycloalkyl. In yet a further embodiment is a compound of Formula (I) or (II) is a compound wherein E is (C═O)—OR, a carboxylic acid bioisostere, —(C═O)—NR¹R, —(C₁-C₇)alkyl-(C═O)—OR, or —(C₁-C₇)alkyl-(C═O)—NR¹R. In one embodiment a compound of Formula (I) or (II) is a compound wherein A is O, B is (C₃-C₈)cycloalkyl, E is (C═O)—OR, and R is H. In a further embodiment, a compound of Formula (I) or (II) is a compound wherein B is cyclohexyl, and R is H. In yet a further embodiment, a compound of Formula (I) or (II) is a compound wherein B is cyclopentyl and R is H. In yet a further embodiment a compound of Formula (I) or (II) is a compound having the following structure:

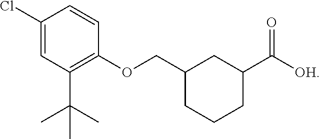 [SMILES: CC(C)(C)c1cc(Cl)ccc1OCC1CCCC(C(=O)O)C1]

In another embodiment is a compound of Formula (I) or (II) is a compound having the structure:

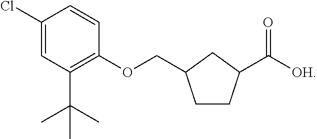 [SMILES: CC(C)(C)c1cc(Cl)ccc1OCC1CCC(C(=O)O)C1]

In one embodiment a compound of Formula (I) or (II) is a compound wherein R is:

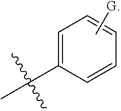

In another embodiment a compound of Formula (I) or (II) is a compound wherein E is (C═O)—OR. In a further embodiment a compound of Formula (I) or (II) is a compound wherein R is H. In yet a further embodiment a compound of Formula (I) or (II) is a compound wherein the compound is selected from the group consisting of: 5-(2-tert-butyl-4-chlorophenoxy)-N-(4-hydroxyphenyl)pentanamide, 7-(2-tert-butyl-4-chlorophenoxy)-N-(4-hydroxyphenyl)heptanamide, 4-(5-(2-tert-butyl-4-chlorophenoxy)pentanamido)benzoic acid, 4-(3-((2-tert-butyl-4-chlorophenoxy)methyl)cyclopentanamido)benzoic acid, 5-(2-tert-butyl-4-chlorophenoxy)pentanoic acid, 4-(2-tert-butyl-4-chlorophenoxy)butanoic acid, 2-(3-((2-tert-butyl-4-chlorophenoxy)methyl)cyclopentyl)acetic acid, 7-(2-tert-butyl-4-chlorophenoxy)heptanoic acid, 4-(5-(2-tert-butyl-4-chlorophenoxy)pentanamido)benzamide, 3-((2-tert-butyl-4-chlorophenoxy)methyl)cyclohexanecarboxylic acid, 3-((2-tert-butyl-4-chlorophenoxy)methyl)cyclopentanecarboxylic acid, 3-((2-tert-butyl-4-chlorophenylamino)methyl)cyclopentanamide, 4-(3-((2-tert-butyl-4-chlorophenoxy)methyl)cyclopentanecarboxamido)benzoic acid, and 5-(2-tert-butyl-4-chlorophenylthio)pentanoic acid. Other compounds of Formula (I) and/or (II) are set forth in PCT/US08/76499, filed Sep. 16, 2008, and published as WO 2009/042444, which is hereby incorporated by reference for such compounds.
In some embodiments, an individual in need of a treatment described herein is an individual diagnosed with elevated levels of plasma retinol, plasma RBP4 (apo-RBP) or plasma RBP4-retinol (holo-RBP). In certain embodiments, an individual in need of a treatment described herein is a male human individual having plasma RBP4 concentration that is greater than 25 μg/mL and/or an apo-RBP-to-holo-RBP ratio above 0.5. In some embodiments, an individual in need of a treatment described herein is a female human individual having plasma RBP4 concentration that is greater than 20 μg/mL and/or an apo-RBP-to-holo-RBP ratio above 0.25. In certain embodiments, an individual in need of a treatment described herein is an individual diagnosed with excessive lipofuscin accumulation, a macular dystrophy, Stargardt's disease, or AMD.
In certain embodiments, any method described herein further comprises diagnosing the individual in need thereof with elevated tissue levels of plasma retinol, plasma RBP4 (apo-RBP), plasma RBP4-retinol (holo-RBP), or an apo-RBP-to-holo-RBP ratio above 0.5. In some embodiments, any method described herein further comprises diagnosing the individual in need thereof with elevated circulating Holo-RBP levels or a circulating apo-RBP-to-holo-RBP ratio above 0.5. In certain embodiments, any method described herein further comprises diagnosing an individual with an increased risk of skin cancer by diagnosing the individual with GA, non-exudative AMD or exudative AMD.
In some embodiments, an effective amount of a therapeutic agent (e.g., N-(4-hydroxyphenyl)retinamide) is an amount sufficient to reduce circulating vitamin A in the individual by between 25% and 75%. In certain embodiments, an effective amount of a therapeutic agent (e.g. N-(4-hydroxyphenyl)retinamide) is an amount sufficient to reduce circulating Holo-RBP by between 25% and 75%. In some embodiments, an effective amount of a therapeutic agent (e.g., N-(4-hydroxyphenyl)retinamide) a therapeutic agent thereof is an amount sufficient to reduce the sum concentration of circulating apo-RBP or holo-RBP by between 25% and 75%. In some embodiments, an effective amount of a therapeutic agent (e.g., N-(4-hydroxyphenyl)retinamide) is less than 300 mg daily. In certain embodiments, an effective amount of a therapeutic agent (e.g., N-(4-hydroxyphenyl)retinamide) is about 50 mg to about 150 mg daily.
In some embodiments, the skin cancer of any method described herein is a skin carcinoma. In some embodiments, the skin cancer of any method described herein is a basal cell carcinoma, squamous cell carcinoma, or melanoma.
In certain embodiments, an effective amount of a therapeutic agent (e.g., N-(4-hydroxyphenyl)retinamide) thereof is initially administered in a loading dose schedule and subsequently administered in a maintenance dose schedule. In some embodiments, the loading dose schedule comprises administering a therapeutic agent (e.g., N-(4-hydroxyphenyl)retinamide) in an amount and period sufficient to reduce circulating vitamin A in the individual by between 25% and 75% and the maintenance dose is administered in an amount sufficient to maintain the reduction of circulating vitamin A. In certain embodiments, the loading dose schedule comprises administering a therapeutic agent (e.g., N-(4-hydroxyphenyl)retinamide) in an amount and period sufficient to reduce circulating holo-RBP in the individual by between 25% and 75% and the maintenance dose is administered in an amount sufficient to maintain the reduction of circulating holo-RBP. In some embodiments, the loading dose schedule comprises administering N-(4-hydroxyphenyl)retinamide, N-(4-methoxyphenyl)retinamide, 4-oxo-N-(4-hydroxyphenyl)retinamide, or a combination thereof in an amount and period sufficient to reduce sum concentration of circulating apo-RBP and holo-RBP in the individual by between 25% and 75% and the maintenance dose is administered in an amount sufficient to maintain the reduction of the sum concentration of circulating apo-RBP and holo-RBP. In certain embodiments, the loading dose schedule comprises administering N-(4-hydroxyphenyl)retinamide, N-(4-methoxyphenyl)retinamide, 4-oxo-N-(4-hydroxyphenyl)retinamide, or a combination thereof in an amount and period sufficient to achieve either a circulating or tissue apo-RBP-to-holo-RBP ratio at about 0.5 and the maintenance dose is administered in an amount sufficient to maintain either circulating or tissue apo-RBP-to-holo-RBP ratio at about 0.5.

Provided herein, in some embodiments, are methods of reducing the incidences of or prophylaxis of conjunctival malignant melanoma (CMM) in an individual in need thereof or an individual diagnosed with GA or AMD comprising administering to the individual an effective amount of a therapeutic agent(s), the therapeutic agent(s) being N-(4-hydroxyphenyl)retinamide, N-(4-methoxyphenyl)retinamide, 4-oxo-N-(4-hydroxyphenyl)retinamide, a compound of Formula (I):

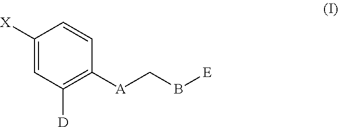

wherein:
A is O, NH, or S;
B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
D is isopropyl, isobutyl, sec-butyl, tert-butyl, neopentyl, sec-pentyl, isopentyl, cyclopropyl, cyclobutyl, cyclopentyl, methylenecyclopropyl, methylenecyclobutyl, methylenecyclopentyl;
E is (C═O)—OR, —O—(C═O)—R, —(C═O)—R, —OR, a carboxylic acid bioisostere, —(C═O)—NR¹R, NR¹—(C═O)—R, —(C₁-C₇)alkyl-(C═O)—OR, or —(C₁-C₇)alkyl-(C═O)—NR¹R;
R is H or

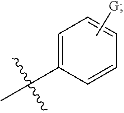

G is —OR¹, —(C₁-C₆)alkyl, —(C₁-C₆)alkyl-OR¹, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
X is a halogen;
   or an active metabolite, or a pharmaceutically acceptable prodrug, salt, or solvate thereof;
   or a combination thereof.

Disclosed herein, in certain embodiments, are methods of treating a non-melanoma skin cancer in an individual diagnosed with AMD comprising administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the non-melanoma skin cancer is basal cell carcinoma or squamous cell carcinoma. In some embodiments, the active agent is a retinoid or a retinoid derivative. In some embodiments, the active agent is N-(4-hydroxyphenyl) retinamide, N-(4-methoxyphenyl)retinamide, 4-oxo-N-(4-hydroxyphenyl)retinamide, a compound of Formula (I):

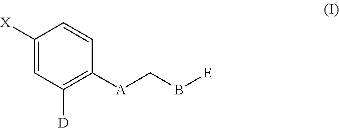

wherein:
A is O, NH, or S;
B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
D is isopropyl, isobutyl, sec-butyl, tert-butyl, neopentyl, sec-pentyl, isopentyl, cyclopropyl, cyclobutyl, cyclopentyl, methylenecyclopropyl, methylenecyclobutyl, methylenecyclopentyl;
E is (C═O)—OR, —O—(C═O)—R, —(C═O)—R, —OR, a carboxylic acid bioisostere, —(C═O)—NR¹R, NR¹—(C═O)—R, —(C₁-C₇)alkyl-(C═O)—OR, or —(C₁-C₇)alkyl-(C═O)—NR¹R;
R is H or

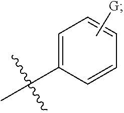

G is —OR¹, —(C₁-C₆)alkyl, —(C₁-C₆)alkyl-OR¹, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
X is a halogen;
   or an active metabolite, or a pharmaceutically acceptable prodrug, salt, or solvate thereof;
   or a combination thereof.

Disclosed herein, in certain embodiments, are methods of inhibiting the recurrence of a non-melanoma skin cancer in an individual diagnosed with AMD, comprising administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the non-melanoma skin cancer is basal cell carcinoma or squamous cell carcinoma. In some embodiments, the active agent is a retinoid or a retinoid derivative. In some embodiments, the active agent is N-(4-hydroxyphenyl) retinamide, N-(4-methoxyphenyl)retinamide, 4-oxo-N-(4-hydroxyphenyl)retinamide, a compound of Formula (I):

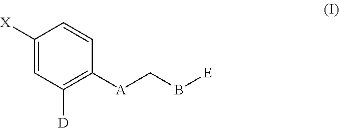

wherein:
A is O, NH, or S;
B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
D is isopropyl, isobutyl, sec-butyl, tert-butyl, neopentyl, sec-pentyl, isopentyl, cyclopropyl, cyclobutyl, cyclopentyl, methylenecyclopropyl, methylenecyclobutyl, methylenecyclopentyl;
E is (C═O)—OR, —O—(C═O)—R, —(C═O)—R, —OR, a carboxylic acid bioisostere, —(C═O)—NR¹R, NR¹—(C═O)—R, —(C₁-C₇)alkyl-(C═O)—OR, or —(C₁-C₇)alkyl-(C═O)—NR¹R;
R is H or;

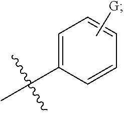

G is —OR¹, —(C₁-C₆)alkyl, —(C₁-C₆)alkyl-OR¹, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
X is a halogen;
   or an active metabolite, or a pharmaceutically acceptable prodrug, salt, or solvate thereof;
   or a combination thereof.

Disclosed herein, in certain embodiments, are methods of treating Gorlin's Syndrome, comprising administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the active agent is a retinoid or a retinoid derivative. In some embodiments, the active agent is N-(4-hydroxyphenyl) retinamide, N-(4-methoxyphenyl)retinamide, 4-oxo-N-(4-hydroxyphenyl)retinamide, a compound of Formula (I):

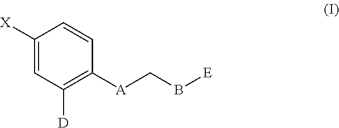

wherein:
A is O, NH, or S;
B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
D is isopropyl, isobutyl, sec-butyl, tert-butyl, neopentyl, sec-pentyl, isopentyl, cyclopropyl, cyclobutyl, cyclopentyl, methylenecyclopropyl, methylenecyclobutyl, methylenecyclopentyl;
E is (C═O)—OR, —O—(C═O)—R, —(C═O)—R, —OR, a carboxylic acid bioisostere, —(C═O)—NR¹R, NR₁—(C═O)—R, —(C₁-C₇)alkyl-(C═O)—OR, or —(C₁-C₇)alkyl-(C═O)—NR¹R;
R is H or

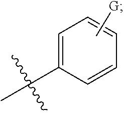

G is —OR¹, —(C₁-C₆)alkyl, —(C₁-C₆)alkyl-OR¹, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
X is a halogen;
   or an active metabolite, or a pharmaceutically acceptable prodrug, salt, or solvate thereof;
   or a combination thereof.

Disclosed herein, in certain embodiments, are methods of treating a basal cell carcinoma in an individual diagnosed with Gorlin's Syndrome, comprising administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the active agent is a retinoid or a retinoid derivative. In some embodiments, the active agent is N-(4-hydroxyphenyl) retinamide, N-(4-methoxyphenyl)retinamide, 4-oxo-N-(4-hydroxyphenyl)retinamide, a compound of Formula (I):

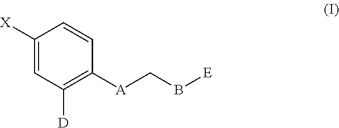

wherein:
A is O, NH, or S;
B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
D is isopropyl, isobutyl, sec-butyl, tert-butyl, neopentyl, sec-pentyl, isopentyl, cyclopropyl, cyclobutyl, cyclopentyl, methylenecyclopropyl, methylenecyclobutyl, methylenecyclopentyl;
E is (C═O)—OR, —O—(C═O)—R, —(C═O)—R, —OR, a carboxylic acid bioisostere, —(C═O)—NR¹R, NR¹—(C═O)—R, —(C₁-C₇)alkyl-(C═O)—OR, or —(C₁-C₇)alkyl-(C═O)—NR¹R;
R is H or

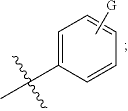

G is —OR¹, —(C₁-C₆)alkyl, —(C₁-C₆)alkyl-OR¹, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
X is a halogen;
   or an active metabolite, or a pharmaceutically acceptable prodrug, salt, or solvate thereof;
   or a combination thereof.

Disclosed herein, in certain embodiments, are methods of inhibiting the recurrence of a basal cell carcinoma in an individual diagnosed with Gorlin's Syndrome, comprising administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the active agent is a retinoid or a retinoid derivative. In some embodiments, the active agent is N-(4-hydroxyphenyl) retinamide, N-(4-methoxyphenyl)retinamide, 4-oxo-N-(4-hydroxyphenyl)retinamide, a compound of Formula (I):

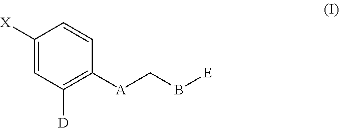

wherein:
A is O, NH, or S;
B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
D is isopropyl, isobutyl, sec-butyl, tert-butyl, neopentyl, sec-pentyl, isopentyl, cyclopropyl, cyclobutyl, cyclopentyl, methylenecyclopropyl, methylenecyclobutyl, methylenecyclopentyl;
E is (C═O)—OR, —O—(C═O)—R, —(C═O)—R, —OR, a carboxylic acid bioisostere, —(C═O)—NR¹R, NR¹—(C═O)—R, —(C₁-C₇)alkyl-(C═O)—OR, or —(C₁-C₇)alkyl-(C═O)—NR¹R;
R is H or

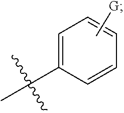

G is —OR¹, —(C₁-C₆)alkyl, —(C₁-C₆)alkyl-OR¹, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
X is a halogen;
   or an active metabolite, or a pharmaceutically acceptable prodrug, salt, or solvate thereof;
   or a combination thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the incidence of Treatment Emergent choroidal neovascularization (CNV) in a phase 2 clinical trial which evaluated the efficacy of HPR to treat GA.

FIG. 2 illustrates the effects of HPR on VEGF expression in fetal human retinal pigment epithelium cultures.

FIG. 3 illustrates the incidences of treatment emergent cancers in HPR GA trial.

FIG. 4 illustrates the effect of HPR on melanoma cell line (B16(RL)).

FIG. 5 illustrates the effect of HPR and compounds of Formula II on two human melanoma cell lines (M207 and SK-MEL28).

FIG. 6 illustrates the effect of HPR on pentazocine binding to Sigma 1 receptor.

FIG. 7 illustrates the effect of HPR competition with pentazocine for binding to Sigma 1 receptor.

FIG. 8 illustrates that fenretinide up-regulates certain complement genes (Crry, CFH, MCP-1, CD59a, Daf2, and CD59b) in the eye of BALC/C**mice. Bars on the left are the levels of gene activation in mice fed normal chow. Bars on the left are the levels of gene activation in mice fed fenretinide supplemented chow (1 g fenretinide/kg chow). Expression of complement genes was determined by RT-PCR. Data was normalized to expression of 18S RNA.

FIG. 9 illustrates that high-dose fenretinide down-regulates certain complement genes (Crry, CFH, MCP-1, CD59a, Daf2, and CD59b) in the eye of BALC/C**mice. Bars on the left are the levels of gene activation in mice fed normal chow. Bars on the left are the levels of gene activation in mice fed fenretinide supplemented chow (Ig fenretinide/kg chow). Expression of complement genes was determined by RT-PCR. Data was normalized to expression of 18S RNA.

FIG. 10 illustrates one possible way that fenretinide modulates ceramide biosynthesis.

FIG. 11 is an illustration of how Patched1 (Ptch1) suppresses activation of Smoothened (Smo). In instances where the hedgehog ligand (Hh) is not bound to PTCH1, PTCH1 is active and suppresses activation of SMO. Inactive SMO is unable to activate GLI transcription factors, which mediate cellular growth.

FIG. 12 is an illustration of how Hedgehog (Hh) results in activation of SMO. In instances where the hedgehog ligand (Hh) is bound to PTCH1, PTCH1 is inactivated and proceeds through an endocytic pathway leading to lysosomal degradation. Loss of PTCH1 leads to increased levels of intracellular oxysterols and SMO translocates to the primary cilium where it activates GLI.

FIG. 13 is an illustration of how fenretinide affects the regulation of SMO. In this scenario, Fenretinide, its metabolites, or compounds of Formula I or II, induce the accumulation of ceramide through activation of sphingomyelinase, which hydrolyzes sphingomyelin. Ceramide then displaces cholesterol from the plasma membrane, and intracellular lipid rafts, thereby creating a concentration gradient which results in reduced levels of cholesterol and its oxidized derivatives (oxysterols)(2,3). SMO remains confined to endosomal vesicles.

FIG. 14 is an illustration of how fenretinide affects the regulation of SMO. In this scenario, Fenretinide, its metabolites, or compounds of Formula I or II, induce the synthesis of ceramide through activation of sphingomyelinase. Ceramide then displaces cholesterol from the plasma membrane, and intracellular lipid rafts. Displaced cholesterol then prevents binding of Hh by occupying the cholesterol binding site on PTCH1. Active PTCH1 continues to facilitate the removal of oxysterols. SMO remains confined to endosomal vesicles.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Provided in certain embodiments herein are methods of reducing the incidences of or reducing the likelihood of developing cancer (e.g., skin cancer) in an individual diagnosed with or suspected of having AMD, the method comprising administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the active agent is a retinoid or a derivative thereof. In some embodiments, the active agent is fenretinide (N-(4-hydroxyphenyl)retinamide, HPR), a compound of Formula (I), or a metabolite thereof). Provided in some embodiments herein are methods of prophylactically treating (i.e., prophylaxis of) cancer (e.g., skin cancer) in an individual diagnosed with or suspected of having AMD, the method comprising administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the active agent is a retinoid or a derivative thereof. In some embodiments, the active agent is fenretinide (N-(4-hydroxyphenyl)retinamide, HPR), a compound of Formula (I), or a metabolite thereof).

Provided in certain embodiments herein are methods of reducing the incidences of or reducing the likelihood of developing a non-melanoma skin cancer (e.g. a basal cell carcinoma or a squamous cell carcinoma) in an individual diagnosed with or suspected of having AMD, the method comprising administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the active agent is a retinoid or a derivative thereof. In some embodiments, the active agent is fenretinide (N-(4-hydroxyphenyl)retinamide, HPR), a compound of Formula (I), or a metabolite thereof). Provided in some embodiments herein are methods of prophylactically treating (i.e., prophylaxis of) a non-melanoma skin cancer (e.g. a basal cell carcinoma or a squamous cell carcinoma) in an individual diagnosed with or suspected of having AMD, the method comprising administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the active agent is a retinoid or a derivative thereof. In some embodiments, the active agent is fenretinide (N-(4-hydroxyphenyl)retinamide, HPR), a compound of Formula (I), or a metabolite thereof).

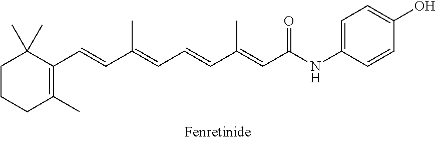 [SMILES: CC1=C(/C=C/C(C)=C/C=C/C(C)=C/C(=O)Nc2ccc(O)cc2)C(C)(C)CCC1]

Provided in certain embodiments herein are methods of reducing the incidences of or reducing the likelihood of developing a basal cell carcinoma in an individual diagnosed with or suspected of having AMD, the method comprising administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the active agent is a retinoid or a derivative thereof. In some embodiments, the active agent is fenretinide (N-(4-hydroxyphenyl)retinamide, HPR), a compound of Formula (I), or a metabolite thereof). Provided in some embodiments herein are methods of prophylactically treating (i.e., prophylaxis of) a basal cell carcinoma in an individual diagnosed with or suspected of having AMD, the method comprising administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the active agent is a retinoid or a derivative thereof. In some embodiments, the active agent is fenretinide (N-(4-hydroxyphenyl)retinamide, HPR), a compound of Formula (I), or a metabolite thereof).

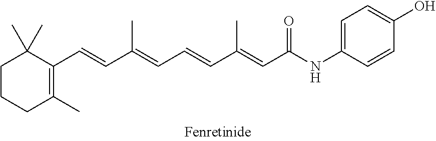 [SMILES: CC1=C(/C=C/C(C)=C/C=C/C(C)=C/C(=O)Nc2ccc(O)cc2)C(C)(C)CCC1]

Provided in certain embodiments herein are methods of reducing the incidences of or reducing the likelihood of developing a squamous cell carcinoma in an individual diagnosed with or suspected of having AMD, the method comprising administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the active agent is a retinoid or a derivative thereof. In some embodiments, the active agent is fenretinide (N-(4-hydroxyphenyl)retinamide, HPR), a compound of Formula (I), or a metabolite thereof). Provided in some embodiments herein are methods of prophylactically treating (i.e., prophylaxis of) a squamous cell carcinoma an individual diagnosed with or suspected of having AMD, the method comprising administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the active agent is a retinoid or a derivative thereof. In some embodiments, the active agent is fenretinide (N-(4-hydroxyphenyl)retinamide, HPR), a compound of Formula (I), or a metabolite thereof).

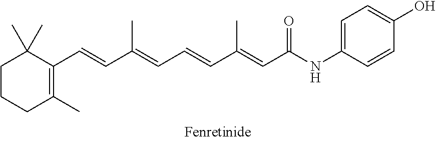 [SMILES: CC1=C(/C=C/C(C)=C/C=C/C(C)=C/C(=O)Nc2ccc(O)cc2)C(C)(C)CCC1]

Provided in certain embodiments herein are methods of reducing the incidences of or reducing the likelihood of developing a basal cell carcinoma in an individual diagnosed with (or suspected of having) Gorlin's Syndrome, the method comprising administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the active agent is a retinoid or a derivative thereof. In some embodiments, the active agent is fenretinide (N-(4-hydroxyphenyl)retinamide, HPR), a compound of Formula (I), or a metabolite thereof). Provided in some embodiments herein are methods of prophylactically treating (i.e., prophylaxis of) a basal cell carcinoma in an individual diagnosed with (or suspected of having) Gorlin's Syndrome, the method administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the active agent is a retinoid or a derivative thereof. In some embodiments, the active agent is fenretinide (N-(4-hydroxyphenyl)retinamide, HPR), a compound of Formula (I), or a metabolite thereof).

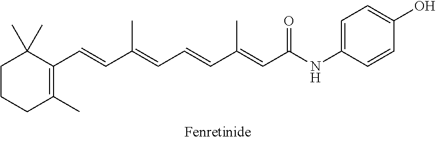 [SMILES: CC1=C(/C=C/C(C)=C/C=C/C(C)=C/C(=O)Nc2ccc(O)cc2)C(C)(C)CCC1]

In certain embodiments, a compound of Formula (I) is:

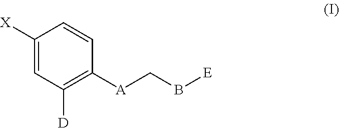

wherein:
A is O, NH, or S;
B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
D is isopropyl, isobutyl, sec-butyl, tert-butyl, neopentyl, sec-pentyl, isopentyl, cyclopropyl, cyclobutyl, cyclopentyl, methylenecyclopropyl, methylenecyclobutyl, methylenecyclopentyl;
E is (C═O)—OR, —O—(C═O)—R, —(C═O)—R, —OR, a carboxylic acid bioisostere, —(C═O)—NR¹R, NR¹—(C═O)—R, —(C₁-C₇)alkyl-(C═O)—OR, or —(C₁-C₇)alkyl-(C═O)—NR¹R;
R is H or

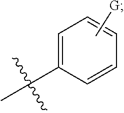

G is —OR¹, —(C₁-C₆)alkyl, —(C₁-C₆)alkyl-OR¹, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
X is a halogen;
   or an active metabolite, or a pharmaceutically acceptable prodrug, salt, or solvate thereof.

In some embodiments, compounds of Formula I include compounds of Formula (II):

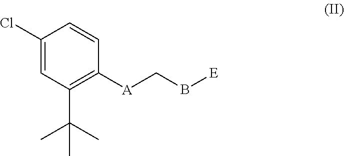

wherein:
A is O, NH, or S;
B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
E is (C═O)—OR, —O—(C═O)—R, —(C═O)—R, —OR, a carboxylic acid bioisostere, —(C═O)—NR¹R, NR¹—(C═O)—R, —(C₁-C₇)alkyl-(C═O)—OR, or —(C₁-C₇)alkyl-(C═O)—NR¹R;
R is H or

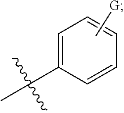

G is —OR¹, —(C₁-C₆)alkyl, —(C₁-C₆)alkyl-OR¹, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
   or an active metabolite, or a pharmaceutically acceptable prodrug, salt, or solvate thereof.

In specific embodiments, a compound of Formula (I) or (II) is a compound wherein A is O. In a further embodiment, a compound of Formula (I) or (II) is a compound wherein B is —(CH₂)ₙ and n is 1-6, or B is —(C₃-C₈)cycloalkyl. In yet a further embodiment is a compound of Formula (I) or (II) is a compound wherein E is (C═O)—OR, a carboxylic acid bioisostere, —(C═O)—NR¹R, —(C₁-C₇)alkyl-(C═O)—OR, or —(C₁-C₇)alkyl-(C═O)—NR¹R. In one embodiment a compound of Formula (I) or (II) is a compound wherein A is O, B is (C₃-C₈)cycloalkyl, E is (C═O)—OR, and R is H. In a further embodiment, a compound of Formula (I) or (II) is a compound wherein B is cyclohexyl, and R is H. In yet a further embodiment, a compound of Formula (I) or (II) is a compound wherein B is cyclopentyl and R is H. In yet a further embodiment a compound of Formula (I) or (II) is a compound having the following structure:

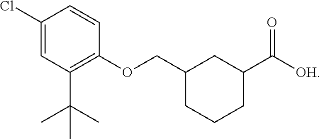 [SMILES: CC(C)(C)c1cc(Cl)ccc1OCC1CCCC(C(=O)O)C1]

In another embodiment is a compound of Formula (I) or (II) is a compound having the structure:

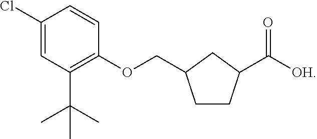 [SMILES: CC(C)(C)c1cc(Cl)ccc1OCC1CCC(C(=O)O)C1]

In one embodiment a compound of Formula (I) or (II) is a compound wherein R is:

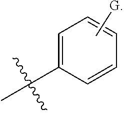

In another embodiment a compound of Formula (I) or (II) is a compound wherein E is (C═O)—OR. In a further embodiment a compound of Formula (I) or (II) is a compound wherein R is H. In yet a further embodiment a compound of Formula (I) or (II) is a compound wherein the compound is selected from the group consisting of: 5-(2-tert-butyl-4-chlorophenoxy)-N-(4-hydroxyphenyl)pentanamide, 7-(2-tert-butyl-4-chlorophenoxy)-N-(4-hydroxyphenyl)heptanamide, 4-(5-(2-tert-butyl-4-chlorophenoxy)pentanamido)benzoic acid, 4-(3-((2-tert-butyl-4-chlorophenoxy)methyl)cyclopentanamido)benzoic acid, 5-(2-tert-butyl-4-chlorophenoxy)pentanoic acid, 4-(2-tert-butyl-4-chlorophenoxy)butanoic acid, 2-(3-((2-tert-butyl-4-chlorophenoxy)methyl)cyclopentyl)acetic acid, 7-(2-tert-butyl-4-chlorophenoxy)heptanoic acid, 4-(5-(2-tert-butyl-4-chlorophenoxy)pentanamido)benzamide, 3-((2-tert-butyl-4-chlorophenoxy)methyl)cyclohexanecarboxylic acid, 3-((2-tert-butyl-4-chlorophenoxy)methyl)cyclopentanecarboxylic acid, 3-((2-tert-butyl-4-chlorophenylamino)methyl)cyclopentanamide, 4-(3-((2-tert-butyl-4-chlorophenoxy)methyl)cyclopentanecarboxamido)benzoic acid, and 5-(2-tert-butyl-4-chlorophenylthio)pentanoic acid. Other compounds of Formula (I) and/or (II) are set forth in PCT/US08/76499, filed Sep. 16, 2008, and published as WO 2009/042444, which is hereby incorporated by reference for such compounds.

In some embodiments, a method described herein is useful for reducing the incidences of, reducing the likelihood of developing, or prophylactically treating (i.e., a method of prophylaxis of) a skin cancer selected from, by way of non-limiting example, basal cell carcinoma, squamous cell carcinoma, melanoma, or the like. In certain embodiments, an individual treated according to any method described herein has been diagnosed with or is suspected of having AMD. In certain embodiments, an individual treated according to any method described herein has a skin cancer, or is diagnosed as having an increased likelihood of developing skin cancer (e.g., due to a diagnosis of AMD). In some embodiments, the embodiments, an individual that has or is diagnosed as having an increased likelihood of developing skin cancer is an individual having or diagnosed as having excessive lipofuscin accumulation, a macular dystrophy, Stargardt's disease, GA, non-exudative AMD, exudative AMD, elevated retinol levels, elevated levels of apo- or holo-RBP, elevated Sigma receptor levels (e.g., elevated Sigma-1 and/or Sigma-2 receptor levels), elevated VEGF levels, and/or an elevated ratio of apo- to holo-RBP.

Provided in some embodiments herein are methods of reducing the incidences of cancer (e.g., skin cancer) in an individual in need thereof comprising administering to the individual an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the active agent is a retinoid or a derivative thereof. In some embodiments, the active agent is 4-HPR, 4-MPR, 4-oxo-4-HPR, a compound of Formula (I), a Sigma receptor modulator, a Sigma-1 receptor modulator, a Sigma-2 receptor modulator, a dual agent that reduces serum retinol-RBP and modulates Sigma receptors (e.g., Sigma-1 and/or Sigma-2), dextromethorphan, or a combination thereof.

### Patient Population

In certain embodiments, the inventors have discovered that individuals suffering from certain disorders of the eye suffer from increased incidence of skin cancer (e.g., non-melanoma skin cancers). For example, in certain instances, about 2% of normal human individuals 75 year old and older develop skin cancer, including basal cell carcinoma, squamous cell carcinoma, and melanoma. In some instances, individuals 75 years old or older and suffering from certain types of eye disorders (e.g., age related macular degeneration, geographic atrophy, or the like) develop skin cancer at a frequency twice (or more) that of normal.

In some embodiments, individuals treated according to a method described herein suffer from and/or have been diagnosed with excessive lipofuscin accumulation, a macular dystrophy, Stargardt's disease, GA, non-exudative AMD, and/or exudative AMD. In certain embodiments, a method described herein further comprises diagnosing an individual with excessive lipofuscin accumulation, a macular dystrophy, Stargardt's disease, GA, non-exudative AMD, and/or exudative AMD.

In certain embodiments, an individual treated according to any method described herein has or has been diagnosed with elevated retinol levels. Elevated retinol levels include, for example, elevated circulating retinol levels and/or elevated tissue retinol levels. In certain embodiments, elevated retinol levels include circulating levels of greater than 2 mol/L. In some embodiments, elevated retinol levels include circulating levels of greater than 2.5 mol/L. In certain embodiments, elevated retinol levels include circulating levels of greater than 3 mol/L. In certain embodiments, elevated retinol levels include circulating levels of greater than 3.5 mol/L. In some embodiments, elevated retinol levels include circulating levels of greater than 4 mol/L. In certain embodiments, elevated retinol levels include circulating levels of greater than 5 mol/L. In some embodiments, a method described herein further comprises diagnosing an individual with elevated retinol levels. In specific embodiments, an individual is diagnosed with increased retinol levels by obtaining a sample from an individual (e.g., a tissue sample, a serum sample, a plasma sample, or the like) and measuring the amount of retinol present in the sample.

In some embodiments, an individual treated according to any method described herein has or has been diagnosed with elevated levels of RBP4. Elevated RBP4 levels include, for example, elevated circulating retinol levels and/or elevated tissue retinol levels. In certain embodiments, elevated RBP levels include circulating levels of RBP4 that are greater than 20 μg/mL. In certain embodiments, elevated RBP levels include circulating levels of RBP4 that are greater than 21 μg/mL. In certain embodiments, elevated RBP levels include circulating levels of RBP4 that are greater than 22 μg/mL. In certain embodiments, elevated RBP levels include circulating levels of RBP4 that are greater than 23 μg/mL. In certain embodiments, elevated RBP levels include circulating levels of RBP4 that are greater than 24 μg/mL. In some embodiments, elevated RBP levels include circulating levels of RBP4 that are greater than 25 μg/mL. In certain embodiments, elevated RBP levels include circulating levels of RBP4 that are greater than 30 μg/mL. In certain embodiments, elevated RBP levels include circulating levels of RBP4 that are greater than 35 μg/mL. In some embodiments, elevated RBP levels include circulating levels of RBP4 that are greater than 40 μg/mL. In certain embodiments, elevated RBP levels include circulating levels of RBP4 that are greater than 50 μg/mL. In certain embodiments, elevated RBP levels include circulating levels of RBP4 that are between 25 μg/mL and 100 μg/mL. In specific embodiments, the individual is a male having a circulating RBP4 level of greater than 25 μg/mL. In more specific embodiments, the individual is a male human having a circulating RBP4 level of greater than 26 μg/mL. In still more specific embodiments, the individual is a male human having a circulating RBP4 level of greater than 27 μg/mL. In yet more specific embodiments, the individual is a male human having a circulating RBP4 level of greater than, greater than 28 μg/mL. In more specific embodiments, the individual is a male human having a circulating RBP4 level of greater than 29 μg/mL. In yet more specific embodiments, the individual is a male human having a circulating RBP4 level of greater than 30 μg/mL. In certain embodiments, elevated RBP levels include circulating levels of RBP4 that are between 20 μg/mL and 100 μg/mL. In specific embodiments, the individual is a female having a circulating RBP4 level of greater than 21 μg/mL. In more specific embodiments, the individual is a female human having a circulating RBP4 level of greater than 22 μg/mL. In still more specific embodiments, the individual is a female human having a circulating RBP4 level of greater than 23 μg/mL. In yet more specific embodiments, the individual is a female human having a circulating RBP4 level of greater than, greater than 24 μg/mL. In more specific embodiments, the individual is a female human having a circulating RBP4 level of greater than 25 μg/mL. In yet more specific embodiments, the individual is a female human having a circulating RBP4 level of greater than 26 μg/mL. In some embodiments, a method described herein further comprises diagnosing an individual with elevated RBP levels. In specific embodiments, an individual is diagnosed with increased RBP levels by obtaining a sample from an individual (e.g., a tissue sample, a serum sample, a plasma sample, or the like) and measuring the amount of RBP present in the sample.

In certain embodiments, an individual treated according to any method described herein has or has been diagnosed with elevated ratios (in molar concentration) of apo-RBP to holo-RBP. Elevated ratios of apo-RBP to holo-RBP include, for example, elevated circulating ratios of apo-RBP to holo-RBP and/or elevated tissue ratios of apo-RBP to holo-RBP. In certain embodiments, elevated ratios of apo-RBP to holo-RBP include ratios (e.g., circulating ratios) of greater than 0.5. In some embodiments, elevated ratios of apo-RBP to holo-RBP include ratios (e.g., circulating ratios) of greater than 0.6. In certain embodiments, elevated ratios of apo-RBP to holo-RBP include ratios (e.g., circulating ratios) of greater than 0.7. In certain embodiments, elevated ratios of apo-RBP to holo-RBP include ratios (e.g., circulating ratios) of greater than 0.8. In certain embodiments, elevated ratios of apo-RBP to holo-RBP include ratios (e.g., circulating ratios) of greater than 0.9. In some embodiments, a method described herein further comprises diagnosing an individual with elevated ratios of apo-RBP to holo-RBP. In specific embodiments, an individual is diagnosed with elevated ratios of apo-RBP to holo-RBP by obtaining a sample from an individual (e.g., a tissue sample, a serum sample, a plasma sample, or the like) and measuring the ratio of apo-RBP to holo-RBP present in the sample.

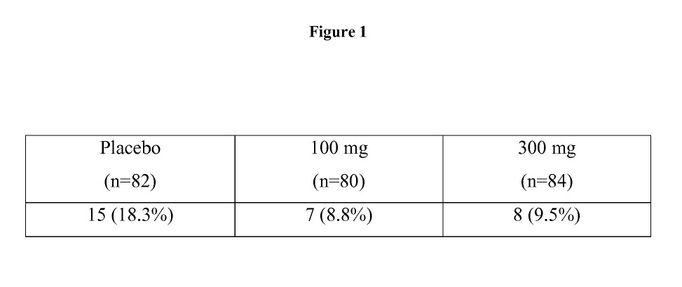

In certain embodiments, an individual treated according to any method described herein has or has been diagnosed with elevated or over-expressed levels of Sigma receptors or activated Sigma receptors (e.g., Sigma-1 and/or Sigma-2 receptors). Elevated levels of sigma receptors include, for example, elevated circulating levels of sigma receptors and/or elevated tissue or eye levels of sigma receptors. In some instances, elevated levels of sigma receptors include levels wherein abnormal neovascularization or retinal cell death occurs (i.e., death of Muller cells, nerve fiber cells, ganglion cells, cells of the inner or outer plexiform layer, photoreceptor cells, or cells of the pigmented epithelium). FIG. 1 illustrates the effect of HPR on incidence of Treatment Emergent choroidal neovascularization (CNV) in a phase 2 clinical trial which evaluated the efficacy of HPR to treat GA.

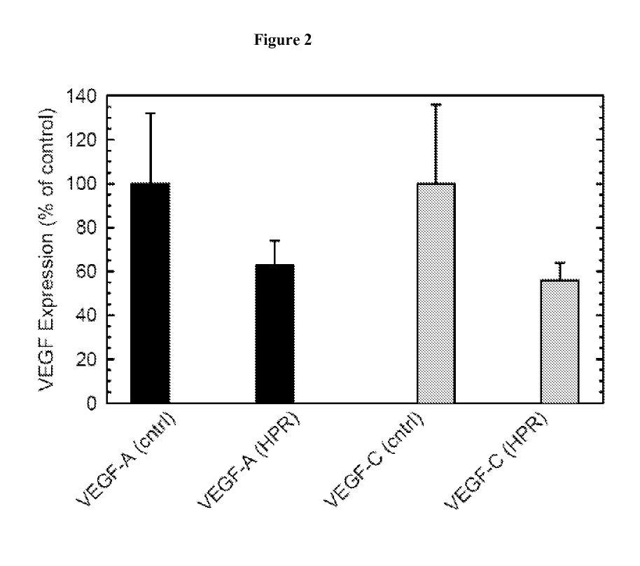

In certain embodiments, an individual treated according to any method described herein has or has been diagnosed with elevated levels of VEGF (e.g., VEGF-A or VEGF-C). Elevated levels of sigma receptors include, for example, elevated circulating levels of sigma receptors and/or elevated tissue or eye levels of sigma receptors. FIG. 2 illustrates the effects of HPR on VEGF expression in cultures of retinal pigment epithelium prepared from human fetal eyes.

In various embodiments, circulating levels described herein are obtained from, by way of non-limiting example, serum samples, plasma samples, or the like. In some embodiments, tissue samples are obtained from, by way of non-limiting example, fat tissue, or the like.

### Indications

### Complement-Mediated Skin Cancers

Disclosed herein, in certain embodiments, are methods of treating complement-mediated skin cancers. By way of non-limiting example, complement-mediated skin cancers include basal cell carcinomas and squamous cell carcinomas.

In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway treats a complement-mediated skin cancer in an individual in need thereof. In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway reduces the recurrence of complement-mediated skin cancer. In some embodiments, the individual has been diagnosed with or is suspected of having AMD. In some embodiments, the active agent is a retinoid or a derivative thereof. In some embodiments, the active agent is fenretinide (N-(4-hydroxyphenyl)retinamide, HPR), a compound of Formula (I), or a metabolite thereof).

### Basal Cell Carcinoma

Basal cell carcinoma is the most common type of skin cancer. Approximately 900,000 cases of basal cell carcinoma are diagnosed each year. The estimated lifetime risk of basal cell carcinoma in Caucasians is 33-39% in men and 23-28% in women. Approximately 35% of individuals that develop at least one basal cell carcinoma will experience recurrence within 3 years. The recurrence rate at 5 years is about 50%. While rarely fatal, it may cause significant destruction or deformation of the skin.

In certain instances, basal cell carcinomas result from (partially or fully) the formation of thymine dimers in the basal cells of the skin. In some embodiments, a thymine dimer in a basal cell results in a gain-of-function mutation in SMO or a loss-of-function mutation in PTCH1.

In certain instances, basal cell carcinomas result from (partially or fully) a gain-of-function mutation in SMO and/or a loss-of-function mutation in PTCH1.

In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway treats a basal cell carcinoma in an individual in need thereof. See, Table 1 and Example 3.

TABLE 1

| | Placebo | 100 mg fenretinide | 300 mg fenretinide |
|---|---|---|---|
| | (n = 82) | (n = 80) | (n = 84) |
| All neoplasms | 14 (17.1%) | 4 (5.0%) | 10 (11.9%) |
| Basal Cell | 5 (6.1%) | 2 (2.5%) | 3 (3.6%) |
| Carcinoma | | | |

In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway reduces the recurrence of basal cell carcinomas. In some embodiments, the individual has been diagnosed with or is suspected of having AMD. In some embodiments, the active agent is a retinoid or a derivative thereof. In some embodiments, the active agent is fenretinide (N-(4-hydroxyphenyl)retinamide, HPR), a compound of Formula (I), or a metabolite thereof).

Nodular basal cell carcinoma is the most common variety. It frequently appears on the head, neck, and upper back. A nodular basal cell carcinoma presents with at least one of the following characteristics: (a) waxy papules with central depression; (b) a pearly appearance; (c) erosion or ulceration; (d) bleeding; (e) crusting; (f) rolled borders; (g) translucency; and (h) telangiectases over the surface.

Pigmented basal cell carcinoma presents with many of the same characteristics of nodular basal cell carcinoma; however, pigmented basal cell carcinomas have increased brown or black pigmentation.

Cystic basal cell carcinoma presents with translucent blue-gray cystic nodules that mimic the appearance of benign cystic lesions.

Micronodular BCC is an aggressive BCC subtype that presents with: (a) waxy papules with central depression; (b) a pearly appearance that appears yellow-white when stretched; (c) bleeding; (d) crusting; (e) well-define, rolled borders; (f) translucency; (g) telangiectases over the surface; and (h) firmness to the touch.

Morpheaform and infiltrating basal cell carcinoma presents with sclerotic (scar-like) plaques or papules. The border of the carcinoma is usually ill defined and often extends well beyond clinical margins. Ulceration, bleeding, and crusting are uncommon. It is often similar in appearance to scar tissue.

### Squamous Cell Carcinoma

Squamous cell carcinoma is the second most common cancer of the skin. It is responsible for about 20% of skin malignancies. The rate of metastasis with squamous cell cancer is low (about 2%-6%), but is higher than the rate associated with basal cell carcinoma.

In certain instances, squamous cell carcinomas result from (partially or fully) the formation of pyrimidine dimers in epidermal keratinocytes. In some embodiments, a pyrimidine dimer in an epidermal keratinocyte results in a gain-of-function mutation in SMO or a loss-of-function mutation in PTCH1.

In certain instances, squamous cell carcinomas result from (partially or fully) a gain-of-function mutation in SMO and/or a loss-of-function mutation in PTCH1.

In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway treats a squamous cell carcinoma in an individual in need thereof. In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway reduces the recurrence of squamous cell carcinomas. In some embodiments, the individual has been diagnosed with or is suspected of having AMD. In some embodiments, the active agent is a retinoid or a derivative thereof. In some embodiments, the active agent is fenretinide (N-(4-hydroxyphenyl)retinamide, HPR), a compound of Formula (I), or a metabolite thereof).

Squamous cell carcinoma in situ (SCC is) presents with atypia involving the full thickness of the epidermis but without invasion into the dermis. Lesions of squamous cell carcinoma in situ range from a scaly pink patch to a thin keratotic papule or plaque.

Typical squamous cell carcinoma presents as a raised, firm, pink-to-flesh-colored keratotic papule or plaque. It most often appears on sun-exposed skin. Skin surface changes may include scaling, ulceration, crusting, or the presence of a cutaneous horn.

Periungual squamous cell carcinoma is similar in appearance to a verruca. Less commonly, lesions may resemble chronic paronychia with swelling, erythema, and tenderness of the nail fold; onychodystrophy also may be noted.

Marjolin ulcer presents as a new area of induration, elevation, or ulceration at the site of a preexisting scar or ulcer.

Perioral squamous cell carcinoma presents on the vermillion border of the lower lip. It appears as a papule, erosion, or focus of erythema/induration. Intraoral squamous cell carcinoma typically manifests as a white plaque (leukoplakia) with or without reddish reticulation (erythroplakia). Common locations include the anterior floor of the mouth, the lateral tongue, and the buccal vestibule.

Verrucous carcinoma presents as exophytic, fungating, verrucous nodules or plaques, which may be described as “cauliflower-like.”

### Gorlin's Syndrome

Gorlin's Syndrome (also known as, Basal Cell Carcinoma Syndrome) is a disease characterized by multiple anatomical deformations and a predisposition to the development of basal cell carcinoma. Gorlin's Syndrome results in multiple basal cell carcinomas, odontogenic keratocysts, intracranial calcification, bifid ribs, kyphoscoliosis, early calcification of falx cerebri, frontal and temporopariental bossing, hypertelorism, mandibular prognathism, and combinations thereof.

In certain instances, Gorlin's Syndrome results from loss of function mutations in PTCH1, gain of function mutations in SMO, or a combination thereof.

In some embodiments, administering an effective amount of an active agent that decreases the plasma concentration of retinol treats Gorlin's Syndrome in an individual in need thereof. In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway in an individual diagnosed with Gorlin's Syndrome treats a basal cell carcinoma. In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway in an individual diagnosed with Gorlin's Syndrome reduces the recurrence of basal cell carcinomas. In some embodiments, the individual has been diagnosed with or is suspected of having AMD. In some embodiments, the active agent is a retinoid or a derivative thereof. In some embodiments, the active agent is fenretinide (N-(4-hydroxyphenyl)retinamide, HPR), a compound of Formula (I), or a metabolite thereof).

An individual will be diagnosed with Gorlin's Syndrome if they have 2 of the following major criteria, or 1 of the following major criteria and two of the following minor criteria.

### Major Criteria

More than 2 Basal Cell Carcinomas, or 1 Basal Cell Carcinoma in a person younger than 20

Odontogenic keratocysts of the jaw

3 or more palmar or plantar pits

Ectopic calcification or early calcification of the falx cerebri

Bifid, fused or splayed ribs

A first-degree relative diagnosed with Gorlin's Syndrome

### Minor Criteria

Macrocephaly

Congenital malformations (e.g., a cleft palate, frontal bossing, cataracts, colobma, microphtalmia, nystagmus

Skeletal abnormalities (e.g., Sprengel deformity, pectus deformity, polydactyl), syndactyl), hypertelorism)

Radiologic abnormalities (e.g., bridging of the sella turcica, vertebral anomalies, modeling defects, flame-shaped lucencies of hands and feet)

Ovarian and cardio fibroma or medullobalstoma

### Hedgehog Pathway

The hedgehog pathway regulates the transcription of certain genes. In certain instances, malfunctions in the hedgehog pathway result in (partially or fully) the development of cancers. In some embodiments, a malfunction in the hedgehog pathway results in the development of a non-melanoma cancer. In some embodiments, a malfunction in the hedgehog pathway results in the development of a basal cell carcinoma, a squamous cell carcinoma, or a combination thereof. In certain instances, malfunctions in the hedgehog pathway result in (partially or fully) the development of Gorlin's Syndrome.

The hedgehog pathway is initiated by the binding of a hedgehog ligand (Hh; e.g., sonic hedgehog (SHH); desert hedgehog (DHH) and Indian hedgehog (IHH)) to the receptor—Patched-1 (PTCH1). The binding of a hedgehog ligand to PTCH1 results in the inactivation of PTCH1. Inactive PTCH1 results in the activation of the transmembrane protein Smoothened (SMO). The activation of SMO ultimately results in the activation of GLI transcription factors.

In instances where a Hh ligand is not bound to PTCH1, PTCH1 is active. Activation of PTCH1 results in inactivation of SMO. Inactive SMO is unable to activate GLI transcription factors.

In certain instances, loss of function mutations in PTCH1 result in (partially or fully) the constitutive activation of SMO. In certain instances, constitutive activation of SMO results in aberrant transcription of genes involved in cell division. In certain instances, loss of function mutations in PTCH1 result in (partially or fully) the development of a non-melanoma cancer. In certain instances, loss of function mutations in PTCH1 result in (partially or fully] the development of a basal cell carcinoma, a squamous cell carcinoma, or a combination thereof. In certain instances, loss of function mutations in PTCH1 result in (partially or fully] the development of Gorlin's Syndrome.

In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway treats loss of function mutations in PTCH1. In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway in an individual with a loss of function mutation in PTCH1 treats a basal cell carcinoma. In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway in an individual with a loss of function mutation in PTCH1 reduces the recurrence of basal cell carcinomas. In some embodiments, the individual has been diagnosed with or is suspected of having AMD. In some embodiments, the active agent is fenretinide (N-(4-hydroxyphenyl)retinamide, HPR), a compound of Formula (I), or a metabolite thereof).

In certain instances, gain of function mutations in SMO result in (partially or fully) the constitutive activation of SMO. In certain instances, constitutive activation of SMO results in aberrant transcription of genes involved in cell division. In certain instances, gain of function mutations in SMO result in (partially or fully) the development of a non-melanoma cancer. In certain instances, gain of function mutations in SMO result in (partially or fully) the development of a basal cell carcinoma, a squamous cell carcinoma, or a combination thereof. In certain instances, gain of function mutations in SMO result in (partially or fully) the development of Gorlin's Syndrome.

In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway treats gain of function mutations in SMO. In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway in an individual with a gain of function mutation in SMO treats a basal cell carcinoma. In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway in an individual with a gain of function mutation in SMO reduces the recurrence of basal cell carcinomas. In some embodiments, the individual has been diagnosed with or is suspected of having AMD. In some embodiments, the active agent is a retinoid or a derivative thereof. In some embodiments, the active agent is fenretinide (N-(4-hydroxyphenyl)retinamide, HPR), a compound of Formula (I), or a metabolite thereof).

### Ceramide

Disclosed herein, in certain embodiments, are methods of treating cancer in an individual that has been diagnosed with or is suspected of having AMD comprising modulating ceramide biosynthesis.

As used herein, “ceramide” means a molecule that consists of a long-chain or sphingoid base linked to a fatty acid via an amide bond. In certain instances, a ceramide is a lipid second messenger. In certain instances, ceramide biosynthesis results from physiological stress. In certain instances, ceramides are concentrated preferentially into lateral liquid-ordered microdomains (a form of ‘raft’ termed ‘ceramide-rich platforms’). In some embodiments, ceramides displace cholesterol from rafts. In certain instances, ceramides activate PKC.

In some embodiments, increasing ceramide biosynthesis treats a cancer. In some embodiments, the cancer is a complement-associated skin cancer, basal cell carcinoma, or squamous cell carcinoma.

In some embodiments, administering an effective amount of an active agent that increases ceramide levels results in the activation of the complement pathway.

In some embodiments, the active agent is a retinoid or a derivative thereof. In some embodiments, the active agent is fenretinide (N-(4-hydroxyphenyl)retinamide, HPR), a compound of Formula (I), or a metabolite thereof).

In some embodiments, fenretinide increases ceramide biosynthesis in cancerous and pre-cancerous cells. In some embodiments, fenretinide increases ceramide biosynthesis in cancerous and pre-cancerous cells by increasing expression of serine palmitoyltransferase and/or ceramide synthase. See, FIG. 10.

In some embodiments, fenretinide increases ceramide biosynthesis as compared to ceramide biosynthesis in a subject not administered fentrtinide. In some embodiments, ceramides induce the activation of PKC. In some embodiments, PKC induces the activation of complement. Thus, in some embodiments, administration of fenretinide results in the activation of complement.

### Complement Pathway

Disclosed herein, in certain embodiments, are methods of treating cancer in an individual that has been diagnosed with or is suspected of having AMD comprising modulating complement. In some embodiments, the method of treating cancer in an individual that has been diagnosed with or is suspected of having AMD comprises modulating Rbp (retinol binding protein) binding of retinol and complement.

In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway results in the activation of the complement pathway. In some embodiments, administering an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway results in (i) a decrease in the ability of RBP to bind retinol, and (ii) the activation of the complement pathway.

In some embodiments, the cancer is a complement-associated cancer, basal cell carcinoma, or squamous cell carcinoma.

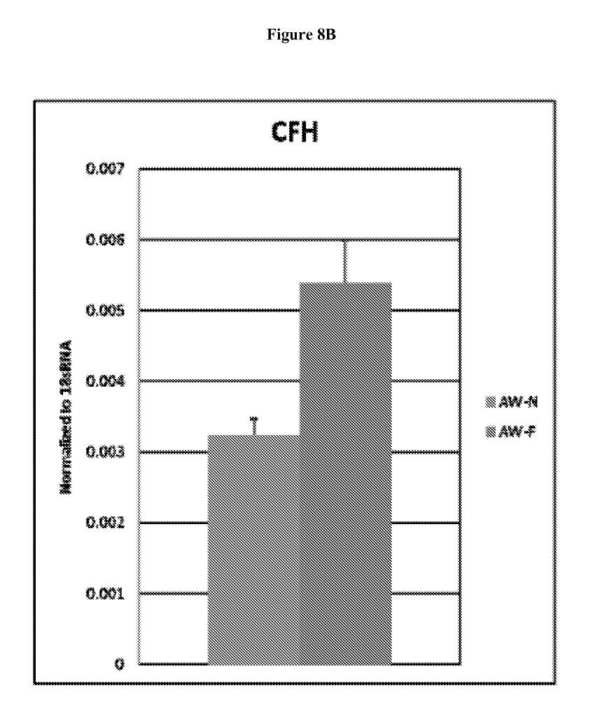

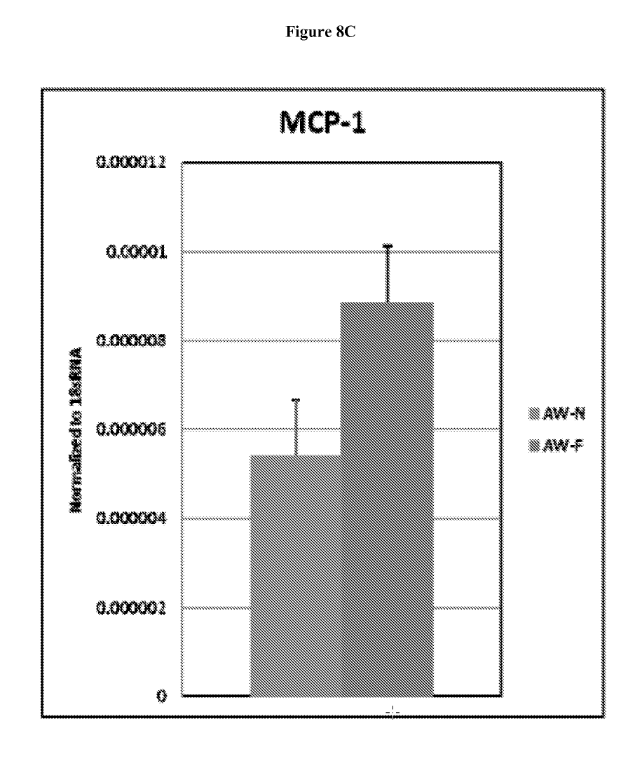

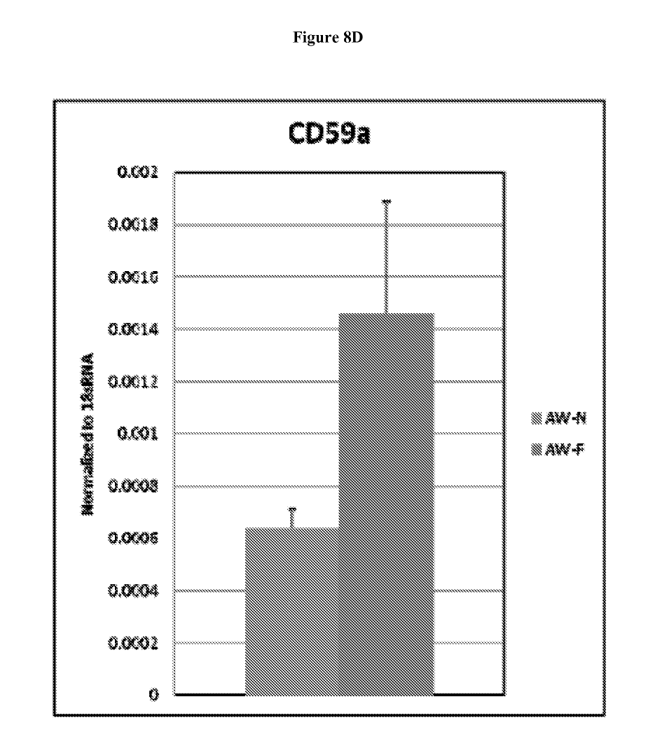

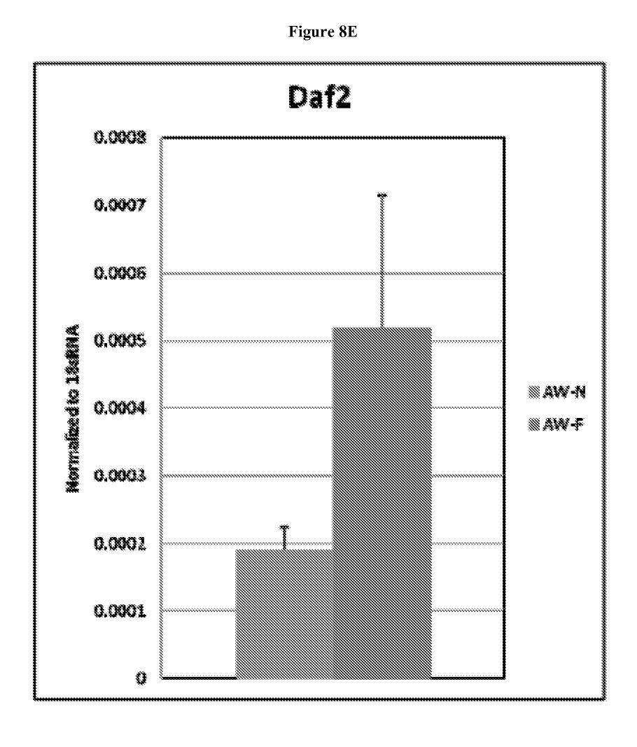

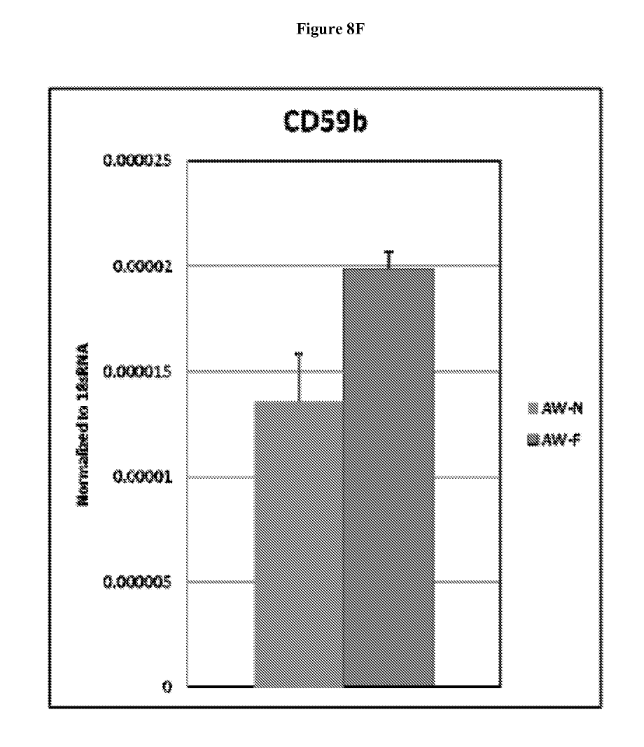

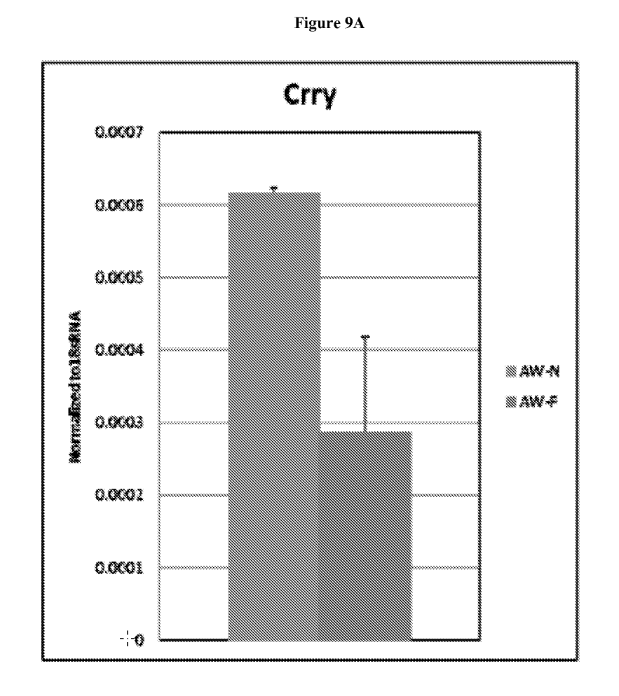

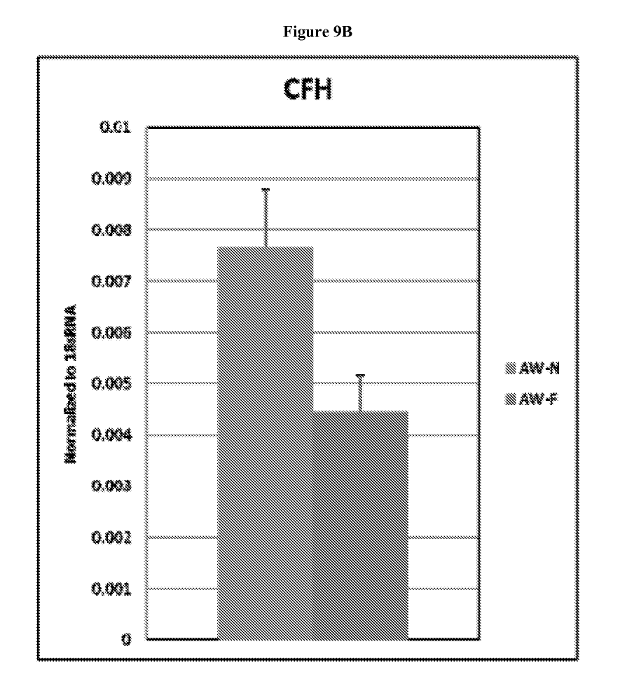

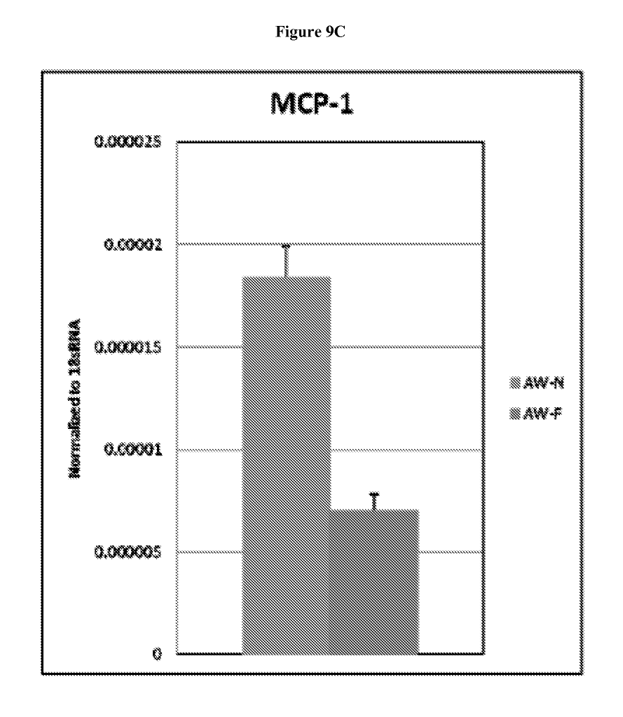

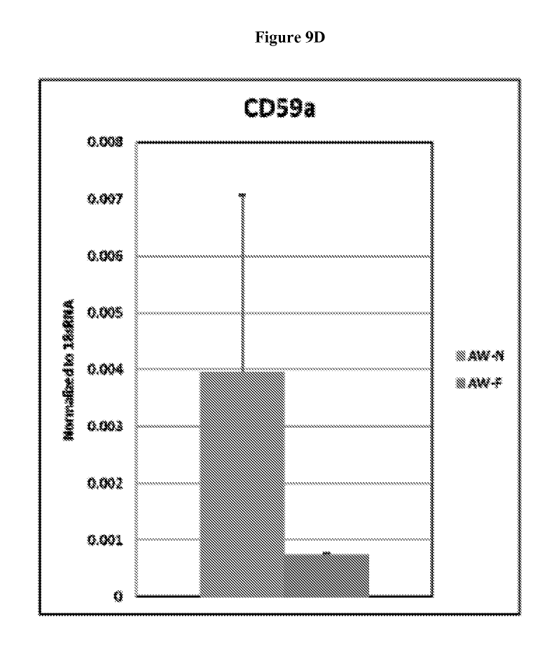

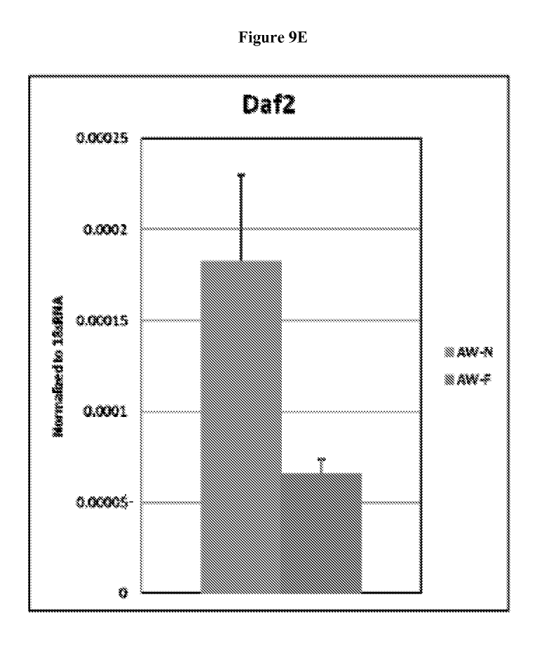

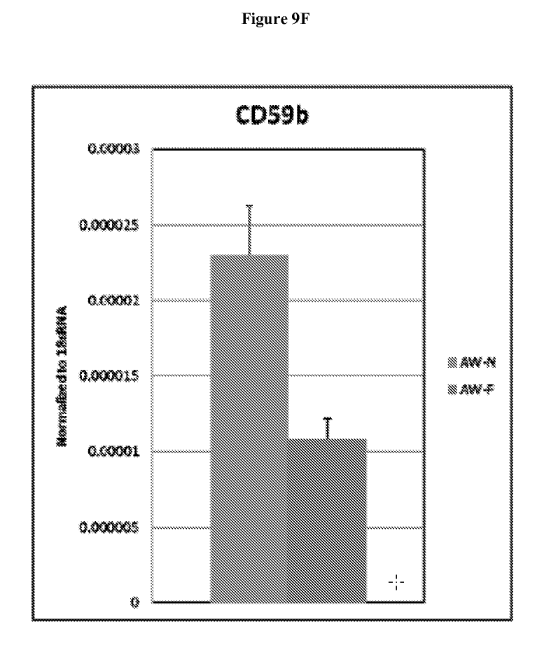

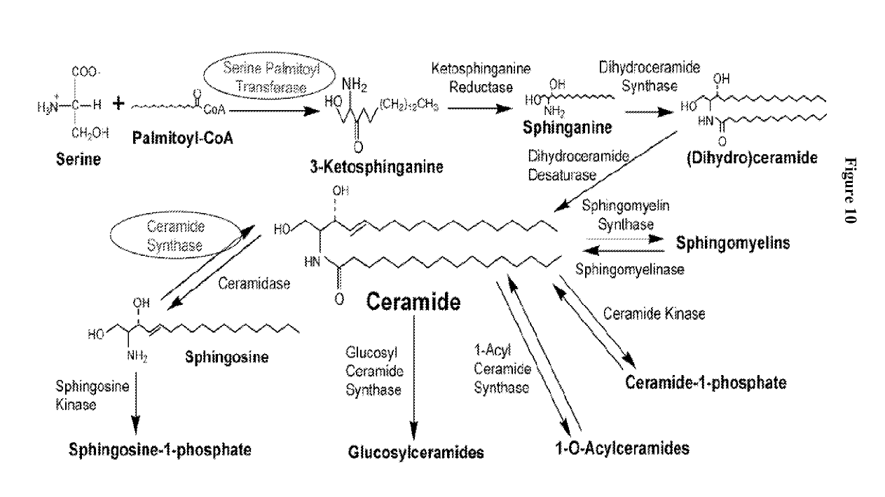

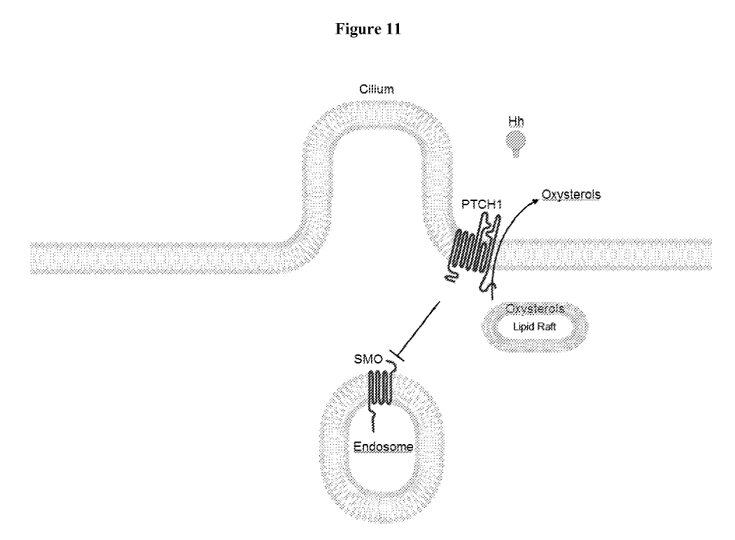

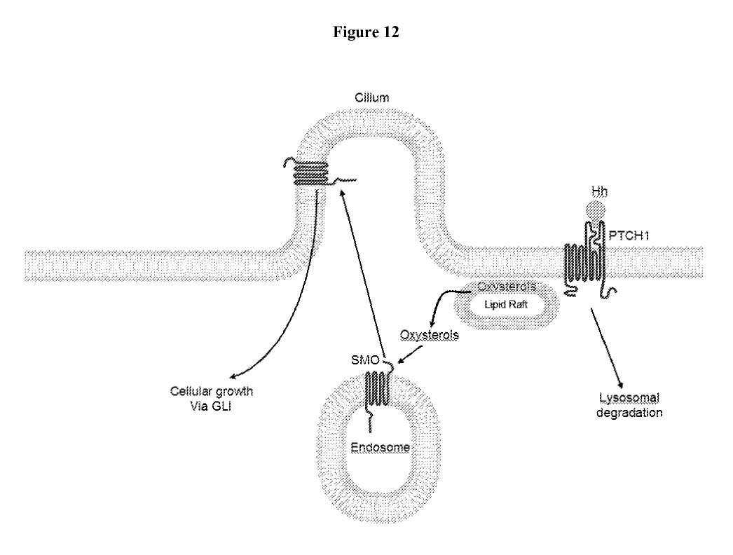

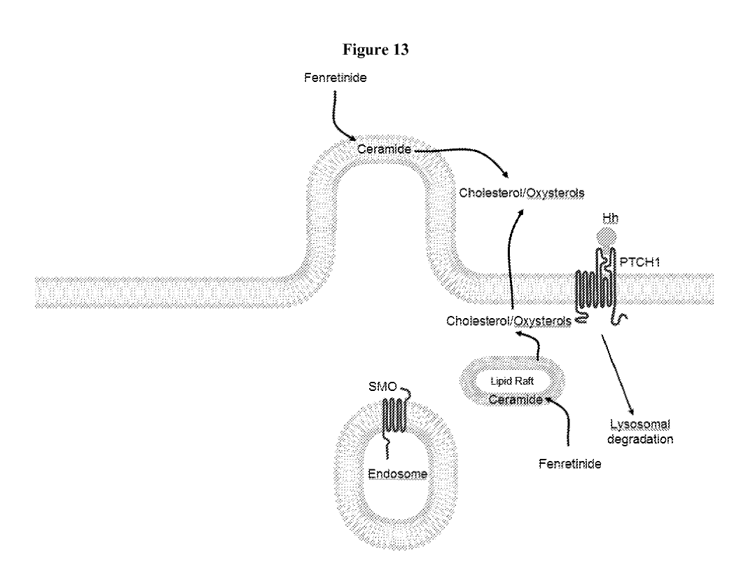

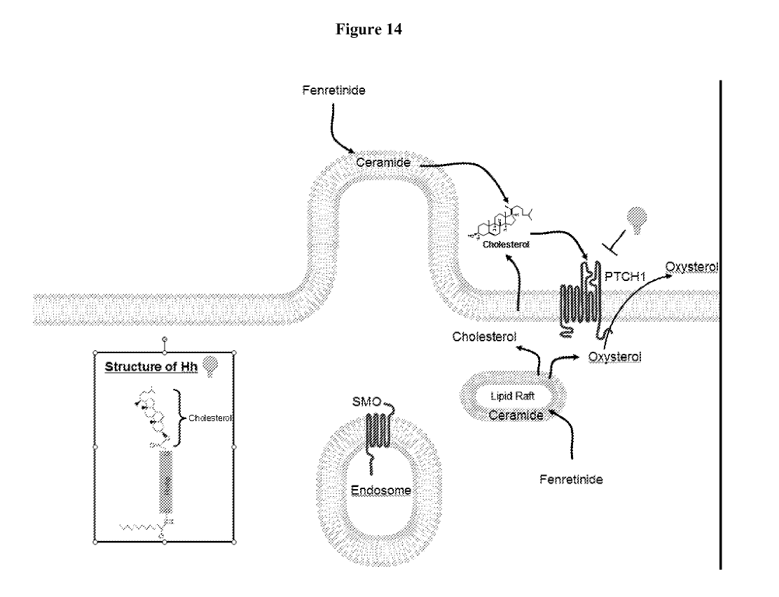

In some embodiments, the active agent is a retinoid or a derivative thereof. In some embodiments, the active agent is fenretinide (N-(4-hydroxyphenyl)retinamide, HPR), a compound of Formula (I), or a metabolite thereof). For example, FIGS. 8 and 9, demonstrate that administration of fenretinide effects the expression of certain complement cascade genes. Low dose fenretinide results in the up-regulation of Crry, CFH, MCP-1, CD59a, CD59b, and Daf2. High dose fenretinide results in the down-regulation of Crry, CFH, MCP-1, CD59a, CD59b, and Daf2.

The complement system is part of the innate immune system. It attacks pathogens in a non-specific manner (i.e., in a non-adaptive manner). In certain instances, the complement system functions by recruiting immune system cells (e.g. macrophages and neutrophils) to the site of an infection by chemotaxis. In certain instances, the complement system also utilizes the complement cascade to attack pathogens and recruit immune system cells. In certain instances, the complement system also removes foreign substances by action of white blood cells (e.g. neutrophils and macrophages).

In certain instances, the inactive complement system comprises over 20 proteins and enzymes, most of which are present in an inactive form. In certain instances, activation of complement (e.g., by the presence of antibodies, the presence of antigens, or the spontaneous hydrolysis of C3) activates the inactive proteases in the system. In certain instances, the proteases cleave targets (e.g., C3). In certain instances, the initial cleavage of the targets results (either partially or fully) in a cascade of cleavages. In certain instances, an active complement system comprises anaphylatoxins (e.g. C3a and C5a), the membrane attack complex (MAC), and proteins that facilitate opsonization (e.g. C3b).

In certain instances, the complement system is activated by three pathways; the classical pathway, the alternative pathway, and the mannose-binding lectin pathway.

In certain instances, the classical pathway begins with the activation of the enzyme C1 (C1q2C1r2C1s). In certain instances, the C1q subunit of this enzyme either directly binds to an antigen or it binds to an antibody bound to an antigen. In certain instances, the binding of C1q leads to a conformational change in C1q. In certain instances, the conformational change in C1q leads to the activation of the two C1r subunits (C1r*) and C1s subunits (C1s*). In certain instances, the activation of the C1r and C1s subunits results in an active C1 enzyme (C1q2C1r*2C1s*).

In certain instances, activated C1 cleaves the protein complement component 4 (C4) into C4a and C4b. In certain instances, C4b binds to the plasma membrane of a pathogen or a host cell. In certain instances, activated C1 also cleaves the protein complement component 2 (C2) into C2a and C2b. In certain instances, C2a binds to C4b forming a C3 convertase (C4bC2a). In certain instances, C4bC2a cleaves the protein C3 into C3a and C3b. In certain instances, C3b binds to the membrane of a pathogen or host cell facilitating opsonization (e.g. the promotion of leukocyte chemoattraction, antigen binding, and phagocytosis) of the pathogen or host cell. In certain instances, C3b binds to C4bC2a forming a C5 convertase (C4bC2aC3a) which cleaves complement component 5 (C5) into C5a and C5b.

In certain instances, the alternative pathway begins with the spontaneous hydrolysis of protein C3 (complement component 3), forming C3(H₂O). In certain instances, the hydrolysis of C3 causes a conformational change that allows Factor B to bind to C3(H₂O). In certain instances, Factor D cleaves Factor B into Ba and Bb. In certain instances, Bb remains bound to C3(H₂O) forming the complex C3(H₂O)Bb (the fluid phase C3 convertase). In certain instances, the fluid phase C3 convertase cleaves C3 into C3a and C3b. In certain instances, C3b binds to the plasma membrane of a pathogen or a host cell where it facilitates opsonization of the host cell or pathogen. In certain instances, the C3b is bound by Factor B. In certain instances, when bound to C3b, Factor B is cleaved by Factor D into Ba and Bb. In certain instances, Bb remains bound to C3b forming an unstable C3 protease (C3Bb). In certain instances, the unstable C3bBb protease is stabilized by the binding of the protein properdin (P) forming a more stable C3 convertase (C3bBbP). In certain instances, upon the binding of a second C3b component, the C3bBbP becomes a C5 convertase (C3bBbC3bP).

In certain instances, C3b binds to the membrane of an antigen presenting cell. In certain instances, the binding of C3b to an antigen presenting cell facilitates opsonization of the antigen presenting cell. In certain instances, the binding of C3b to an antigen presenting cell interferes with ADCC. In some embodiments, the methods described herein comprise depleting and/or inhibiting the activity of a C3 convertase (e.g., C4bC2a, C3(H₂O)Bb, C3bBb, C3bBbP). In some embodiments, the methods described herein comprise inhibiting the formation of a C3 convertase. In some embodiments, depleting and/or inhibiting the activity of a C3 convertase, or inhibiting the formation of a C3 convertase comprises inhibiting the expression of C3, inhibiting the expression of any of the subunits of C1, inhibiting the activity of C1, inhibiting the expression of C4, inhibiting the expression of C2, inhibiting the expression of Factor B, increasing the expression of Factor I, administering exogenous Factor I, administering exogenous CR1.

In certain instances, the Membrane Attack Complex (MAC) comprises five protein sub-units: C5b, C6, C7, C8, and C9. In certain instances, C5b is produced by the cleavage of C5. In certain instances, following the cleavage of C5, C5b binds C6. C5bC6 is then bound by C7. In certain instances, the binding of C7 induces a conformational change in C7, exposing a hydrophobic domain. In certain instances, the hydrophobic domain enables C7 to insert itself into the plasma membrane of a pathogen or host cell. In certain instances, C8 binds to the C5bC6C7 complex. In certain instances, the binding of C8 also induces a conformational change in C8, exposing a hydrophobic domain that enables C8 to insert itself into the plasma membrane. In certain instances, the C5bC6C7C8 complex induces the polymerization of multiple C9 proteins. In certain instances, the C9 proteins form a pore in a plasma membrane. In certain instances, the pore allows the free diffusion of fluids, ions, and proteins into and out of the cell; a process that ultimately leads to the death of a cell.

C3a, C4a, and C5a are anaphylatoxins. In certain instances, anaphylatoxins are fluid phase proteins that bind to receptors on mast cells. In certain instances, anaphylatoxins regulate smooth muscle spasms (e.g. bronchospasms), increase in the permeability of capillaries, and are chemotactic targets of leukocytes (e.g. the follow the increasing concentration gradient of an anaphylatoxin). In certain instances, C3a and C5a are the most potent anaphylatoxins.

In certain instances, C3a regulates degranulation of Mast-cells and serves as a chemotactic target for eosinophile granulocytes.

In certain instances, C5a serves as a chemotactic target for granulocytes and macrophages, and regulates vascular permeability, smooth muscle spasms and mast cell degranulation. In certain instance, C5a accelerates the growth of a neoplasm. In certain instances, C5a recruits myeloid-derived suppressor cells (MDSC). In certain instances, MDSC inhibit (either partially or fully) the activity of CD8⁺ T-cells. In certain instances, antagonizing C5a and/or C5aR inhibits (either partially or fully) the growth of a neoplasm. In some embodiments, the methods described herein deplete and/or inhibit the complement cascade without or only minimally cleaving C5 (i.e., producing C5a).

Disclosed herein, in certain embodiments, are methods of screening an individual to determine efficacy of a compound disclosed herein (e.g., N-(4-hydroxyphenyl)retinamide, N-(4-methoxyphenyl)retinamide, 4-oxo-N-(4-hydroxyphenyl)retinamide, or a compound of Formula (I)) in the treatment of a cancer, comprising determining the expression levels of a gene in the complement cascade. In some embodiments, the gene is selected from: Crry, CFH, MCP-1, CD59a, CD59b, and Daf2. In some embodiments, an increase in the expression of the gene in the complement cascade (e.g., Crry, CFH, MCP-1, CD59a, CD59b, and Daf2) as compared to a normal expression profile for the gene indicates that administration of a compound disclosed herein is efficacious in the treatment of a cancer. In some embodiments, the normal expression profile is determined by measuring the expression level of a complement cascade gene (e.g., Crry, CFH, MCP-1, CD59a, CD59b, and Daf2) in an individual not administered a compound herein (e.g., N-(4-hydroxyphenyl)retinamide, N-(4-methoxyphenyl)retinamide, 4-oxo-N-(4-hydroxyphenyl)retinamide, or a compound of Formula (I)). In some embodiments, the cancer is a complement-associated skin cancer. In some embodiments, the cancer is basal cell carcinoma. In some embodiments, the cancer is squamous cell carcinoma. I

### Active Agents

Provided in certain embodiments herein are methods of treating (a) cancer in an individual diagnosed with or suspected of having AMD or (b) Gorlin's Syndrome, the method comprising administering to an individual in need thereof an effective amount of an active agent that (a) modulates (e.g., decreases) serum retinol; (b) modulates (e.g., increases) ceramide levels; (c) modulates (e.g., decreases the activity of or blocks) a sigma receptor; and/or (d) modulates (e.g., decreases the activity of or blocks) the patched or smoothened receptor within the hedgehog pathway. In some embodiments, the active agent (a) decreases the plasma concentration of retinol, or (b) inhibits the binding of retinol and RBP.

Any retinoid compound, or any derivative of a retinoid, may be used in the methods disclosed herein. In some embodiments, the active agent is a retinoid. In some embodiments, the active agent is a first generation retinoid. In some embodiments, the active agent is a second generation retinoid. In some embodiments, the active agent is a third generation retinoid. In some embodiments, the retinoid is retinol, retinal, tretinoin, isotretinoin, alitretinoin, etretinate, acitretin, tazarotene, bexarotene, adapalene. In some embodiments, the active agent is a derivative of a retinoid. In some embodiments, the active agent is fenretinide, a compound of Formula (I), or a metabolite thereof.

Fenretinide or other therapeutic agents administered in any method described herein is optionally administered as a fenretinide, a pharmaceutically active metabolite of fenretinide, a pharmaceutically acceptable salt of fenretinide or metabolite thereof, a pharmaceutically acceptable N-oxide of fenretinide or metabolite thereof, a pharmaceutically acceptable prodrug of fenretinide or metabolite thereof, or pharmaceutically acceptable solvate of fenretinide or metabolite thereof.

Metabolites of fenretinide include, by way of non-limiting example, RBP-binding fenretinide metabolites, N-(4-methoxyphenyl)retinamide (4-MPR), 4-oxo-N-(4-hydroxyphenyl)retinamide (4-oxo-fenretinide, 4-oxo-4-HPR), and the like.

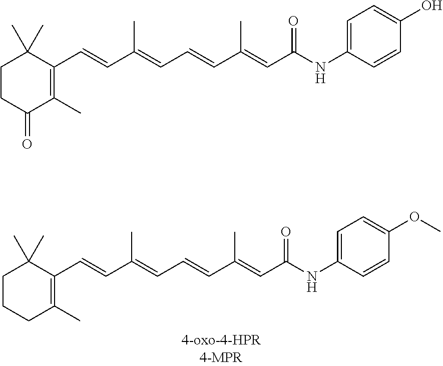 [SMILES: CC1=C(/C=C/C(C)=C/C=C/C(C)=C/C(=O)Nc2ccc(O)cc2)C(C)(C)CCC1=O ; COc1ccc(NC(=O)/C=C(C)/C=C/C=C(C)/C=C/C2=C(C)CCCC2(C)C)cc1]

In some embodiments, the fenretinide or a metabolite thereof may be administered according to any method described herein as a prodrug. A fenretinide prodrug is an agent that is converted into fenretinide or active fenretinide metabolite in vivo. In some embodiments, the prodrug has improved solubility in pharmaceutical compositions over the parent drug. A further example of a prodrug is an ester, carbonate, carbamate, or the like bonded to an alcohol where upon administration to an individual, the prodrug reacts (e.g., through contact with an acidic environment, a metabolic pathway, or the like) to reveal the fenretinide or an active metabolite thereof. Esters include substituted and unsubstituted alkyl esters, substituted and unsubstituted aryl esters, peptides (e.g., comprising and attached through a lysine), amino acid (e.g., lysine), and the like. Carbonates include substituted and unsubstituted alkyl carbonates, substituted and unsubstituted aryl carbonates, and the like. Carbamates include substituted and unsubstituted alkyl carbamates, substituted and unsubstituted aryl carbamates, and the like. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound (e.g., wherein the fenretinide ester is a peptide ester of fenretinide).

Also included in fenretinide or therapeutic agents described herein include compounds of Formula (I), Formula (II), and/or any compound specifically described herein.

An “alkyl” group refers to an aliphatic hydrocarbon group. Alkyl groups include saturated alkyl and/or unsaturated alkyl groups. In some instances, one or more of the carbon atoms of the alkyl group is substituted with a hetero atom. Alkyl groups also include acyclic and cyclic alkyl groups. Acyclic alkyl groups include straight chain and branched alkyl groups.

As used herein, the term “aryl” refers to an aromatic ring, including those wherein each of the atoms forming the ring is a carbon atom and wherein one or more of the atoms forming the ring is a hetero atom (i.e., forming a heteroaryl). Aryl rings disclosed herein include rings having five, six, seven, eight, nine, or more than nine carbon atoms. Examples of aryl groups include, but are not limited to phenyl, and naphthalenyl. Heteroaryl groups are attached to the molecule at any suitable location (e.g., at a heteroatom or at a carbon atom). Illustrative examples of heteroaryl groups include the following moieties:

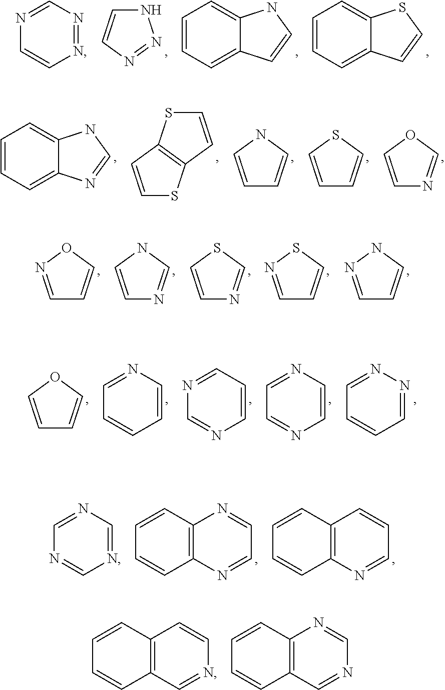

and the like.

### Dosing

In some embodiments, a method for treating any of the diseases or conditions described herein in a subject in need of such treatment, involves administration of a fenretinide or therapeutic agent, e.g., a compound of Formula (I), fenretinide, or a pharmaceutically acceptable salt, pharmaceutically acceptable N-oxide, pharmaceutically active metabolite, pharmaceutically acceptable prodrug, or pharmaceutically acceptable solvate thereof. In certain embodiments, the fenretinide agent is administered in a therapeutically effective amount. In certain embodiments, the fenretinide agent is administered in a pharmaceutical composition comprising the fenretinide agent.

In certain embodiments, the compounds described herein or the compositions thereof are administered for prophylactic treatments. In certain embodiments, amounts effective for theses uses will depend on the severity and course of the disease or condition, previous therapy, the patient's health status, weight, and response to the drugs, and the judgment of the treating physician. In some embodiments, therapeutically effective amounts are determined by any method, including, by way of non-limiting example, by a dose escalation clinical trial.

In certain prophylactic applications, compounds described herein and compositions thereof are administered to a patient susceptible to or otherwise at risk of a particular disease, disorder or condition. In certain embodiments, the effective amount of compound administered is a prophylactically effective amount or dose. In certain embodiments, the precise amounts also depend on the patient's state of health, weight, and the like. In certain embodiments, effective amounts for this use depend on the previous therapy, the patient's health status and response to the drugs, and the judgment of the treating physician. In some embodiments, the effective amounts are determined in any manner, including, e.g., a dose escalation clinical trial.

In some embodiments, doses employed for adult human treatment are in the range of about 10 to about 1000 mg therapeutic or fenretinide agent (e.g., a compound of Formula (I), fenretinide or metabolite thereof) per day. In some embodiments, a method described herein comprises administration of about 20 mg to about 300 mg therapeutic or fenretinide agent (e.g., a compound of Formula (I), fenretinide or metabolite thereof) per day. In certain embodiments, a method described herein comprises administration of less than 300 mg therapeutic or fenretinide agent (e.g., a compound of Formula (I), fenretinide or metabolite thereof) per day. In some embodiments, a method described herein comprises administration of about 40 mg to about 200 mg therapeutic or fenretinide agent (e.g., a compound of Formula (I), fenretinide or metabolite thereof) per day. In certain embodiments, a method described herein comprises administration of about 50 mg to about 150 mg therapeutic or fenretinide agent (e.g., a compound of Formula (I), fenretinide or metabolite thereof) per day. In some embodiments, a method described herein comprises administration of about 80 mg to about 120 mg therapeutic or fenretinide agent (e.g., a compound of Formula (I), fenretinide or metabolite thereof) per day. In certain embodiments, a method described herein comprises administration of about 90 mg to about 110 mg therapeutic or fenretinide agent (e.g., a compound of Formula (I), fenretinide or metabolite thereof) per day. In some embodiments, a method described herein comprises administration of about 100 mg therapeutic or fenretinide agent (e.g., a compound of Formula (I), fenretinide or metabolite thereof) per day. In various embodiments, the desired dose is conveniently presented in a single dose or as divided doses administered simultaneously (or over a short period of time) or at appropriate intervals, for example as two, three, four or more sub-doses per day.

In some embodiments, doses employed for adult human treatment are in the range of about 0.5 mg/kg/day to about 10 mg/kg/day therapeutic or fenretinide agent (e.g., a compound of Formula (I), fenretinide or metabolite thereof). In some embodiments, a method described herein comprises administration of about 0.7 mg/kg/day to about 5 mg/kg/day therapeutic or fenretinide agent (e.g., a compound of Formula (I), fenretinide or metabolite thereof). In certain embodiments, a method described herein comprises administration of less than 5 mg/kg/day therapeutic or fenretinide agent (e.g., a compound of Formula (I), fenretinide or metabolite thereof). In certain embodiments, a method described herein comprises administration of less than 4 mg/kg/day therapeutic or fenretinide agent (e.g., a compound of Formula (I), fenretinide or metabolite thereof). In certain embodiments, a method described herein comprises administration of less than 3 mg/kg/day therapeutic or fenretinide agent (e.g., a compound of Formula (I), fenretinide or metabolite thereof). In certain embodiments, a method described herein comprises administration of less than 2 mg/kg/day therapeutic or fenretinide agent (e.g., a compound of Formula (I), fenretinide or metabolite thereof). In some embodiments, a method described herein comprises administration of about 0.8 mg/kg/day to about 4 mg/kg/day therapeutic or fenretinide agent (e.g., a compound of Formula (I), fenretinide or metabolite thereof). In certain embodiments, a method described herein comprises administration of about 0.9 mg/kg/day to about 3 mg/kg/day therapeutic or fenretinide agent (e.g., a compound of Formula (I), fenretinide or metabolite thereof). In some embodiments, a method described herein comprises administration of about 0.9 mg/kg/day to about 2 mg/kg/day therapeutic or fenretinide agent (e.g., a compound of Formula (I), fenretinide or metabolite thereof). In some embodiments, a method described herein comprises administration of about 1 mg/kg/day to about 2 mg/kg/day therapeutic or fenretinide agent (e.g., a compound of Formula (I), fenretinide or metabolite thereof). In certain embodiments, a method described herein comprises administration of about 1 mg/kg/day to about 1.6 mg/kg/day therapeutic or fenretinide agent (e.g., a compound of Formula (I), fenretinide or metabolite thereof). In some embodiments, a method described herein comprises administration of about 1 mg/kg/day to about 1.5 mg/kg/day therapeutic or fenretinide agent (e.g., a compound of Formula (I), fenretinide or metabolite thereof). In various embodiments, the desired dose is conveniently presented in a single dose or as divided doses administered simultaneously (or over a short period of time) or at appropriate intervals, for example as two, three, four or more sub-doses per day.

In certain embodiments, a method described herein comprises administration of a therapeutic or fenretinide agent (e.g., a compound of Formula (I), fenretinide or metabolite thereof) in a daily amount sufficient to reduce circulating and/or tissue vitamin A in the individual by at least 10%. In some embodiments, a method described herein comprises administration of a therapeutic or fenretinide agent (e.g., a compound of Formula (I), fenretinide or metabolite thereof) in a daily amount sufficient to reduce circulating and/or tissue vitamin A in the individual by at least 25%. In certain embodiments, a method described herein comprises administration of a therapeutic or fenretinide agent (e.g., a compound of Formula (I), fenretinide or metabolite thereof) in a daily amount sufficient to reduce circulating and/or tissue vitamin A in the individual by at least 30%. In certain embodiments, a method described herein comprises administration of a therapeutic or fenretinide agent (e.g., a compound of Formula (I), fenretinide or metabolite thereof) in a daily amount sufficient to reduce circulating and/or tissue vitamin A in the individual by at least 40%. In some embodiments, a method described herein comprises administration of a therapeutic or fenretinide agent (e.g., a compound of Formula (I), fenretinide or metabolite thereof) in a daily amount sufficient to reduce circulating and/or tissue vitamin A in the individual by at least 50%. In certain embodiments, a method described herein comprises administration of a therapeutic or fenretinide agent (e.g., a compound of Formula (I), fenretinide or metabolite thereof) in a daily amount sufficient to reduce circulating and/or tissue vitamin A in the individual by at least 60%. In some embodiments, a method described herein comprises administration of a therapeutic or fenretinide agent (e.g., a compound of Formula (I), fenretinide or metabolite thereof) in a daily amount sufficient to reduce circulating and/or tissue vitamin A in the individual by between 10% and 90%. In some embodiments, a method described herein comprises administration of a therapeutic or fenretinide agent (e.g., a compound of Formula (I), fenretinide or metabolite thereof) in a daily amount sufficient to reduce circulating and/or tissue vitamin A in the individual by between 20% and 80%. In some embodiments, a method described herein comprises administration of a therapeutic or fenretinide agent (e.g., a compound of Formula (I), fenretinide or metabolite thereof) in a daily amount sufficient to reduce circulating and/or tissue vitamin A in the individual by between 25% and 75%. In some embodiments, a method described herein comprises administration of a therapeutic or fenretinide agent (e.g., a compound of Formula (I), fenretinide or metabolite thereof) in a daily amount sufficient to reduce circulating and/or tissue vitamin A in the individual by between 30% and 70%. In certain embodiments, an initial or baseline (e.g., prior to or contemporaneous with initial treatment) and subsequent intermittent (e.g., daily, weekly, monthly, or the like) measurements of vitamin A levels are measured in an individual undergoing any treatment described herein. In further embodiments, these measurements are utilized to adjust and/or titrate the dose of fenretinide agent administered, e.g., so as to achieve the vitamin A levels described herein.

In some embodiments, a method described herein comprises administration of a therapeutic or fenretinide agent (e.g., a compound of Formula (I), fenretinide or metabolite thereof) in a daily amount sufficient to reduce circulating and/or tissue holo-RBP in the individual by at least 10%. In some embodiments, a method described herein comprises administration of a therapeutic or fenretinide agent (e.g., a compound of Formula (I), fenretinide or metabolite thereof) in a daily amount sufficient to reduce circulating and/or tissue holo-RBP in the individual by at least 25%. In certain embodiments, a method described herein comprises administration of a therapeutic or fenretinide agent (e.g., a compound of Formula (I), fenretinide or metabolite thereof) in a daily amount sufficient to reduce circulating and/or tissue holo-RBP in the individual by at least 30%. In certain embodiments, a method described herein comprises administration of a therapeutic or fenretinide agent (e.g., a compound of Formula (I), fenretinide or metabolite thereof) in a daily amount sufficient to reduce circulating and/or tissue holo-RBP in the individual by at least 40%. In some embodiments, a method described herein comprises administration of a therapeutic or fenretinide agent (e.g., a compound of Formula (I), fenretinide or metabolite thereof) in a daily amount sufficient to reduce circulating and/or tissue holo-RBP in the individual by at least 50%. In certain embodiments, a method described herein comprises administration of a therapeutic or fenretinide agent (e.g., a compound of Formula (I), fenretinide or metabolite thereof) in a daily amount sufficient to reduce circulating and/or tissue holo-RBP in the individual by at least 60%. In some embodiments, a method described herein comprises administration of a therapeutic or fenretinide agent (e.g., a compound of Formula (I), fenretinide or metabolite thereof) in a daily amount sufficient to reduce circulating and/or tissue holo-RBP in the individual by between 10% and 90%. In some embodiments, a method described herein comprises administration of a therapeutic or fenretinide agent (e.g., a compound of Formula (I), fenretinide or metabolite thereof) in a daily amount sufficient to reduce circulating and/or tissue holo-RBP in the individual by between 20% and 80%. In some embodiments, a method described herein comprises administration of a therapeutic or fenretinide agent (e.g., a compound of Formula (I), fenretinide or metabolite thereof) in a daily amount sufficient to reduce circulating and/or tissue holo-RBP in the individual by between 25% and 75%. In some embodiments, a method described herein comprises administration of a therapeutic or fenretinide agent (e.g., a compound of Formula (I), fenretinide or metabolite thereof) in a daily amount sufficient to reduce circulating and/or tissue holo-RBP in the individual by between 30% and 70%. In certain embodiments, an initial or baseline (e.g., prior to or contemporaneous with initial treatment) and subsequent intermittent (e.g., daily, weekly, monthly, or the like) measurements of holo-RBP levels are measured in an individual undergoing any treatment described herein. In further embodiments, these measurements are utilized to adjust and/or titrate the dose of fenretinide agent administered, e.g., so as to achieve the holo-RBP levels described herein.

In certain embodiments, a method described herein comprises administration of a therapeutic or fenretinide agent (e.g., a compound of Formula (I), fenretinide or metabolite thereof) in a daily amount sufficient to reduce circulating and/or tissue holo-RBP and free RBP sum concentrations (i.e., the concentration of RBP bound to retinol plus the concentration of free RBP) in the individual by at least 10%. In some embodiments, a method described herein comprises administration of a therapeutic or fenretinide agent (e.g., a compound of Formula (I), fenretinide or metabolite thereof) in a daily amount sufficient to reduce circulating and/or tissue holo-RBP and free RBP sum concentrations in the individual by at least 25%. In certain embodiments, a method described herein comprises administration of a therapeutic or fenretinide agent (e.g., a compound of Formula (I), fenretinide or metabolite thereof) in a daily amount sufficient to reduce circulating and/or tissue holo-RBP and free RBP sum concentrations in the individual by at least 30%. In certain embodiments, a method described herein comprises administration of a therapeutic or fenretinide agent (e.g., a compound of Formula (I), fenretinide or metabolite thereof) in a daily amount sufficient to reduce circulating and/or tissue holo-RBP and free RBP sum concentrations in the individual by at least 40%. In some embodiments, a method described herein comprises administration of a therapeutic or fenretinide agent (e.g., a compound of Formula (I), fenretinide or metabolite thereof) in a daily amount sufficient to reduce circulating and/or tissue holo-RBP and free RBP sum concentrations in the individual by at least 50%. In certain embodiments, a method described herein comprises administration of a therapeutic or fenretinide agent (e.g., a compound of Formula (I), fenretinide or metabolite thereof) in a daily amount sufficient to reduce circulating and/or tissue holo-RBP and free RBP sum concentrations in the individual by at least 60%. In some embodiments, a method described herein comprises administration of a therapeutic or fenretinide agent (e.g., a compound of Formula (I), fenretinide or metabolite thereof) in a daily amount sufficient to reduce circulating and/or tissue holo-RBP and free RBP sum concentrations in the individual by between 10% and 90%. In some embodiments, a method described herein comprises administration of a therapeutic or fenretinide agent (e.g., a compound of Formula (I), fenretinide or metabolite thereof) in a daily amount sufficient to reduce circulating and/or tissue holo-RBP and free RBP sum concentrations in the individual by between 20% and 80%. In some embodiments, a method described herein comprises administration of a therapeutic or fenretinide agent (e.g., a compound of Formula (I), fenretinide or metabolite thereof) in a daily amount sufficient to reduce circulating and/or tissue holo-RBP and free RBP sum concentrations in the individual by between 25% and 75%. In some embodiments, a method described herein comprises administration of a therapeutic or fenretinide agent (e.g., a compound of Formula (I), fenretinide or metabolite thereof) in a daily amount sufficient to reduce circulating and/or tissue holo-RBP and free RBP sum concentrations in the individual by between 30% and 70%. In certain embodiments, an initial or baseline (e.g., prior to or contemporaneous with initial treatment) and subsequent intermittent (e.g., daily, weekly, monthly, or the like) measurements of holo-RBP and free RBP sum concentrations are measured in an individual undergoing any treatment described herein. In further embodiments, these measurements are utilized to adjust and/or titrate the dose of fenretinide agent administered, e.g., so as to achieve the holo-RBP and free RBP sum concentrations described herein.

In certain embodiments, a method described herein comprises administration of a therapeutic or fenretinide agent (e.g., a compound of Formula (I), fenretinide or metabolite thereof) in a daily amount sufficient to provide circulating and/or tissue apo- to holo-RBP ratio (i.e., the ratio of the molar concentration of apo-RBP to the molar concentration of holo-RBP) of about 0.5 to about 1.5. In some embodiments, a method described herein comprises administration of a therapeutic or fenretinide agent (e.g., a compound of Formula (I), fenretinide or metabolite thereof) in a daily amount sufficient to provide circulating and/or tissue apo- to holo-RBP ratio (i.e., the ratio of the molar concentration of apo-RBP to the molar concentration of holo-RBP) of about 0.6 to about 1.4. In certain embodiments, a method described herein comprises administration of a therapeutic or fenretinide agent (e.g., a compound of Formula (I), fenretinide or metabolite thereof) in a daily amount sufficient to provide circulating and/or tissue apo- to holo-RBP ratio (i.e., the ratio of the molar concentration of apo-RBP to the molar concentration of holo-RBP) of about 0.7 to about 1.3. In certain embodiments, a method described herein comprises administration of a therapeutic or fenretinide agent (e.g., a compound of Formula (I), fenretinide or metabolite thereof) in a daily amount sufficient to provide circulating and/or tissue apo- to holo-RBP ratio (i.e., the ratio of the molar concentration of apo-RBP to the molar concentration of holo-RBP) of about 0.8 to about 1.2. In certain embodiments, a method described herein comprises administration of a therapeutic or fenretinide agent (e.g., a compound of Formula (I), fenretinide or metabolite thereof) in a daily amount sufficient to provide circulating and/or tissue apo- to holo-RBP ratio (i.e., the ratio of the molar concentration of apo-RBP to the molar concentration of holo-RBP) of about 0.9 to about 1.1. In certain embodiments, a method described herein comprises administration of a therapeutic or fenretinide agent (e.g., a compound of Formula (I), fenretinide or metabolite thereof) in a daily amount sufficient to provide circulating and/or tissue apo- to holo-RBP ratio (i.e., the ratio of the molar concentration of apo-RBP to the molar concentration of holo-RBP) of about 1. In certain embodiments, an initial or baseline (e.g., prior to or contemporaneous with initial treatment) and subsequent intermittent (e.g., daily, weekly, monthly, or the like) measurements of RBP and retinol concentrations are measured in an individual undergoing any treatment described herein. In further embodiments, these measurements are utilized to adjust and/or titrate the dose of fenretinide agent administered, e.g., so as to achieve the apo- to holo-RBP ratio described herein.

In certain embodiments, a method described herein comprises administration of a therapeutic or fenretinide agent (e.g., a compound of Formula (I), fenretinide or metabolite thereof) in a daily amount sufficient to reduce levels of circulating and/or tissue retinol binding protein (RBP). In some embodiments, a method described herein comprises administration of a therapeutic or fenretinide agent (e.g., a compound of Formula (I), fenretinide or metabolite thereof) in a daily amount sufficient to provide circulating and/or tissue RBP levels of less than 50 μg/mL. In certain embodiments, a method described herein comprises administration of a therapeutic or fenretinide agent (e.g., a compound of Formula (I), fenretinide or metabolite thereof) in a daily amount sufficient to provide circulating and/or tissue RBP levels of less than 40 μg/mL. In some embodiments, a method described herein comprises administration of a therapeutic or fenretinide agent (e.g., a compound of Formula (I), fenretinide or metabolite thereof) in a daily amount sufficient to provide circulating and/or tissue RBP levels of less than 35 μg/mL. In certain embodiments, a method described herein comprises administration of a therapeutic or fenretinide agent (e.g., a compound of Formula (I), fenretinide or metabolite thereof) in a daily amount sufficient to provide circulating and/or tissue RBP levels of less than 30 μg/mL. In some embodiments, a method described herein comprises administration of a therapeutic or fenretinide agent (e.g., a compound of Formula (I), fenretinide or metabolite thereof) in a daily amount sufficient to provide circulating and/or tissue RBP levels of less than 25 μg/mL. In certain embodiments, a method described herein comprises administration of a therapeutic or fenretinide agent (e.g., a compound of Formula (I), fenretinide or metabolite thereof) in a daily amount sufficient to provide circulating and/or tissue RBP levels of less than 20 μg/mL. In some embodiments, a method described herein comprises administration of a therapeutic or fenretinide agent (e.g., a compound of Formula (I), fenretinide or metabolite thereof) in a daily amount sufficient to provide circulating and/or tissue RBP levels of less than 15 μg/mL. In some embodiments, a method described herein comprises administration of a therapeutic or fenretinide agent (e.g., a compound of Formula (I), fenretinide or metabolite thereof) in a daily amount sufficient to provide circulating and/or tissue RBP levels of less than 10 μg/mL.

In certain embodiments, a method described herein comprises administration of a therapeutic or fenretinide agent (e.g., a compound of Formula (I), fenretinide or metabolite thereof) in a daily amount sufficient to reduce levels of circulating and/or tissue retinol binding protein (RBP) complexed with retinol (holo-RBP). In some embodiments, a method described herein comprises administration of a therapeutic or fenretinide agent (e.g., a compound of Formula (I), fenretinide or metabolite thereof) in a daily amount sufficient to provide circulating and/or tissue holo-RBP levels of less than 50 μg/mL. In certain embodiments, a method described herein comprises administration of a therapeutic or fenretinide agent (e.g., a compound of Formula (I), fenretinide or metabolite thereof) in a daily amount sufficient to provide circulating and/or tissue holo-RBP levels of less than 40 μg/mL. In some embodiments, a method described herein comprises administration of a therapeutic or fenretinide agent (e.g., a compound of Formula (I), fenretinide or metabolite thereof) in a daily amount sufficient to provide circulating and/or tissue holo-RBP levels of less than 35 μg/mL. In certain embodiments, a method described herein comprises administration of a therapeutic or fenretinide agent (e.g., a compound of Formula (I), fenretinide or metabolite thereof) in a daily amount sufficient to provide circulating and/or tissue holo-RBP levels of less than 30 μg/mL. In some embodiments, a method described herein comprises administration of a therapeutic or fenretinide agent (e.g., a compound of Formula (I), fenretinide or metabolite thereof) in a daily amount sufficient to provide circulating and/or tissue holo-RBP levels of less than 25 μg/mL. In certain embodiments, a method described herein comprises administration of a therapeutic or fenretinide agent (e.g., a compound of Formula (I), fenretinide or metabolite thereof) in a daily amount sufficient to provide circulating and/or tissue holo-RBP levels of less than 20 μg/mL. In some embodiments, a method described herein comprises administration of a therapeutic or fenretinide agent (e.g., a compound of Formula (I), fenretinide or metabolite thereof) in a daily amount sufficient to provide circulating and/or tissue holo-RBP levels of less than 15 μg/mL. In some embodiments, a method described herein comprises administration of a therapeutic or fenretinide agent (e.g., a compound of Formula (I), fenretinide or metabolite thereof) in a daily amount sufficient to provide circulating and/or tissue holo-RBP levels of less than 10 μg/mL.

In certain embodiments, a method described herein comprises administration of a therapeutic or fenretinide agent (e.g., a compound of Formula (I), fenretinide or metabolite thereof) in a daily amount sufficient to reduce circulating or tissue concentrations or expression of VEGF-A and/or VEGF-C by at least 5%. In some embodiments, a method described herein comprises administration of a therapeutic or fenretinide agent (e.g., a compound of Formula (I), fenretinide or metabolite thereof) in a daily amount sufficient to reduce circulating or tissue concentrations or expression of VEGF-A and/or VEGF-C by at least 10%. In certain embodiments, a method described herein comprises administration of a therapeutic or fenretinide agent (e.g., a compound of Formula (I), fenretinide or metabolite thereof) in a daily amount sufficient to reduce circulating or tissue concentrations or expression of VEGF-A and/or VEGF-C by at least 20%. In certain embodiments, a method described herein comprises administration of a therapeutic or fenretinide agent (e.g., a compound of Formula (I), fenretinide or metabolite thereof) in a daily amount sufficient to reduce circulating or tissue concentrations or expression of VEGF-A and/or VEGF-C by at least 30%. In certain embodiments, a method described herein comprises administration of a therapeutic or fenretinide agent (e.g., a compound of Formula (I), fenretinide or metabolite thereof) in a daily amount sufficient to reduce circulating or tissue concentrations or expression of VEGF-A and/or VEGF-C by at least 40%.

In certain embodiments, a method described herein comprises administration of a therapeutic or fenretinide agent (e.g., a compound of Formula (I), fenretinide or metabolite thereof) in a daily amount sufficient to reduce expression of VEGF-A in the retinal pigment epithelium (RPE) by at least 5%. In some embodiments, a method described herein comprises administration of a therapeutic or fenretinide agent (e.g., a compound of Formula (I), fenretinide or metabolite thereof) in a daily amount sufficient to reduce expression of VEGF-A in the RPE by at least 10%. In certain embodiments, a method described herein comprises administration of a therapeutic or fenretinide agent (e.g., a compound of Formula (I), fenretinide or metabolite thereof) in a daily amount sufficient to reduce expression of VEGF-A in the RPE by at least 20%. In certain embodiments, a method described herein comprises administration of a therapeutic or fenretinide agent (e.g., a compound of Formula (I), fenretinide or metabolite thereof) in a daily amount sufficient to reduce expression of VEGF-A in the RPE by at least 30%. In certain embodiments, a method described herein comprises administration of a therapeutic or fenretinide agent (e.g., a compound of Formula (I), fenretinide or metabolite thereof) in a daily amount sufficient to reduce expression of VEGF-A in the RPE by at least 40%.

In certain embodiments, a method described herein comprises administration of a therapeutic or fenretinide agent (e.g., a compound of Formula (I), fenretinide or metabolite thereof) in a daily amount sufficient to reduce expression of VEGF-C in the retinal pigment epithelium (RPE) by at least 5%. In some embodiments, a method described herein comprises administration of a therapeutic or fenretinide agent (e.g., a compound of Formula (I), fenretinide or metabolite thereof) in a daily amount sufficient to reduce expression of VEGF-C in the RPE by at least 10%. In certain embodiments, a method described herein comprises administration of a therapeutic or fenretinide agent (e.g., a compound of Formula (I), fenretinide or metabolite thereof) in a daily amount sufficient to reduce expression of VEGF-C in the RPE by at least 20%. In certain embodiments, a method described herein comprises administration of a therapeutic or fenretinide agent (e.g., a compound of Formula (I), fenretinide or metabolite thereof) in a daily amount sufficient to reduce expression of VEGF-C in the RPE by at least 30%. In certain embodiments, a method described herein comprises administration of a therapeutic or fenretinide agent (e.g., a compound of Formula (I), fenretinide or metabolite thereof) in a daily amount sufficient to reduce expression of VEGF-C in the RPE by at least 40%.

In certain embodiments, a method described herein comprises administration of a therapeutic or fenretinide agent (e.g., a compound of Formula (I), fenretinide or metabolite thereof) in a daily amount sufficient to inactivate or block at least 5% of circulating or tissue Sigma receptors, such as Sigma-1 and/or Sigma-2 (e.g., through ligand binding of the receptor). In some embodiments, a method described herein comprises administration of a therapeutic or fenretinide agent (e.g., a compound of Formula (I), fenretinide or metabolite thereof) in a daily amount sufficient to inactivate or block at least 10% of circulating or tissue Sigma receptors, such as Sigma-1 and/or Sigma-2 (e.g., through ligand binding of the receptor). In certain embodiments, a method described herein comprises administration of a therapeutic or fenretinide agent (e.g., a compound of Formula (I), fenretinide or metabolite thereof) in a daily amount sufficient to inactivate or block at least 20% of circulating or tissue Sigma receptors, such as Sigma-1 and/or Sigma-2 (e.g., through ligand binding of the receptor). In certain embodiments, a method described herein comprises administration of a therapeutic or fenretinide agent (e.g., a compound of Formula (I), fenretinide or metabolite thereof) in a daily amount sufficient to inactivate or block at least 30% of circulating or tissue Sigma receptors, such as Sigma-1 and/or Sigma-2 (e.g., through ligand binding of the receptor). In certain embodiments, a method described herein comprises administration of a therapeutic or fenretinide agent (e.g., a compound of Formula (I), fenretinide or metabolite thereof) in a daily amount sufficient to inactivate or block at least 40% of circulating or tissue Sigma receptors, such as Sigma-1 and/or Sigma-2 (e.g., through ligand binding of the receptor).

Daily dosing amounts as described herein provide in some embodiments, circulating and/or tissue levels described herein after a single administration or after administration for an extended period of time, e.g., two days, three days, four days, five days, six days, one week, two weeks, three weeks, four weeks, or the like.

In certain embodiments, the amount of a compound administered that corresponds to an effective amount varies depending upon factors such as the identity (e.g., weight and/or age) of the individual in need of treatment, and/or the state of the individual in need of treatment (e.g., RBP circulating and/or tissue levels, holo-RBP circulating and/or tissue levels, and/or retinol circulating and/or tissue levels), etc.

In some embodiments, a fenretinide agent described herein (e.g., fenretinide or a metabolite thereof) is formulated for systemic delivery and/or is administered according to any methods described herein in a manner to achieve systemic delivery. In some embodiments, a fenretinide agent described herein (e.g., fenretinide or a metabolite thereof) is formulated for oral administration and/or is administered according to any methods described herein in an oral manner. In certain embodiments, the pharmaceutical compositions described herein are in unit dosage forms suitable for single administration of precise dosages. In unit dosage form, the formulation is divided into unit doses containing appropriate quantities of one or more compound. In some embodiments, the unit dosage is in the form of a package containing discrete quantities of the formulation. Non-limiting examples are packaged tablets or capsules, and powders in vials or ampoules. In certain embodiments, aqueous suspension compositions are packaged in single-dose non-re-closable containers. In alternative embodiments, multiple-dose reclosable containers are used, in which case it the composition optionally comprises a preservative. In certain embodiments, formulations for parenteral injection are contained within units including, by way of non-limiting example, ampoules, or multi-dose containers. In such embodiments, the formulations/compositions comprise an optional preservative.

### Maintenance Methods

In certain embodiments, a loading dose of fenretinide and/or metabolites thereof is administered to an individual. In certain embodiments, a loading dose is utilized to achieve (1) a therapeutically or prophylactically effective level of circulating and/or tissue therapeutic agent and/or active metabolites thereof in an individual (e.g., levels of a compound of Formula (I), fenretinide levels, fenretinide+metabolite levels, RBP-fenretinide levels, and/or RBP-fenretinide+RBP-fenretinide metabolite levels are increased sufficiently); and/or (2) a therapeutically or prophylactically effective retinol levels (e.g., ratio of apo- to holo-RBP, holo-RBP levels, retinol levels, RBP levels, etc.), or therapeutically or prophylactically effective reduction thereof. In certain embodiments, once a sufficient amount of therapeutic agent, fenretinide and/or active fenretinide metabolites are present in an individual (e.g., the individuals fenretinide levels, fenretinide+metabolite levels, RBP-fenretinide levels, and/or RBP-fenretinide+RBP-fenretinide metabolite levels are increased sufficiently) (e.g., as determined by measuring levels in the individual's serum and/or plasma), holo-RBP levels are decreased sufficiently (e.g., as determined by measuring levels in the individual's serum and/or plasma), unblocked Sigma-receptor levels are sufficiently decreased, or VEGF levels or express is sufficiently decreased, a maintenance dose is administered to substantially maintain such levels. In certain embodiments, the dosage or the frequency of administration, or both, is reduced, as a function of therapeutic agent levels, fenretinide levels, active fenretinide metabolite levels, unblocked Sigma-receptor levels, VEGF levels, and/or holo-RBP levels, such that such levels are substantially retained, or are reduced at a reduced rate. In certain embodiments, patients are given intermittent treatment on a long-term basis upon any spike in levels of therapeutic agent (e.g., levels of a compound of Formula (I), fenretinide levels, active fenretinide metabolite levels, VEGF levels, unblocked Sigma-receptor levels, and/or holo-RBP levels). In certain embodiments, intermittent (e.g., daily, weekly, monthly, or the like) measurements of fenretinide levels, active fenretinide metabolite levels, and/or holo-RBP levels (e.g., serum and/or plasma levels) are obtained from an individual undergoing any treatment described herein. In further embodiments, these intermittent measurements are utilized to adjust and/or titrate the dose administered.

### Sigma Receptor Modulation

Also provided in certain embodiments herein are methods of treating a disease or disorder associated with the over-expression of Sigma receptors (e.g., Sigma-1 and/or Sigma-2) by administering an effective amount of any therapeutic agent described herein (e.g., a compound of Formula (I), fenretinide or metabolite thereof). In certain embodiment, such diseases include, by way of non-limiting example, retinal neurodegeneration, cancer, HIV, stroke, Alzheimer's disease, schizophrenia, Parkinson's disease, or the like, depression, amnesia, and drug addiction. In some embodiments, provided herein are methods of agonizing and/or antagonizing Sigma receptors comprising contacting the Sigma receptors with an effective amount of any agent described herein (e.g., a compound of Formula (I), fenretinide or metabolite thereof).

### EXAMPLE

### Example 1

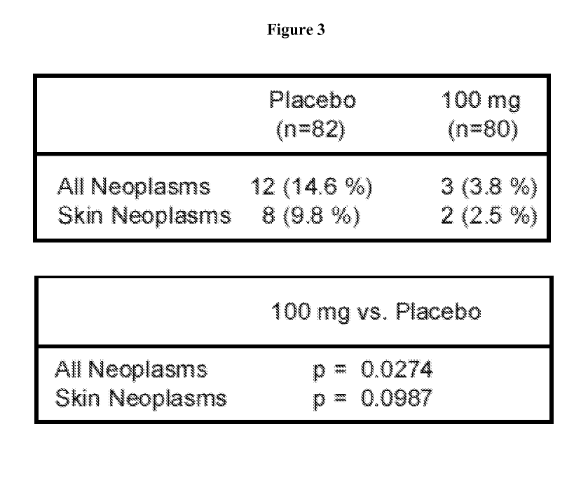

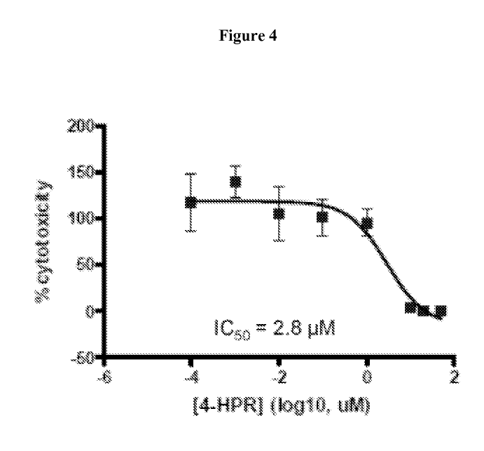

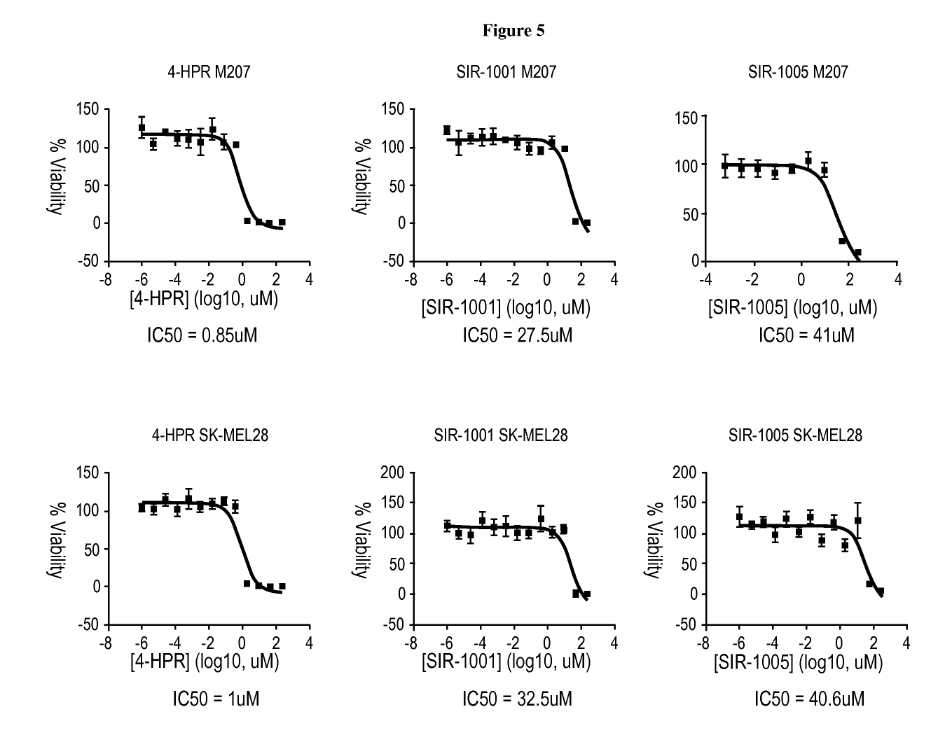

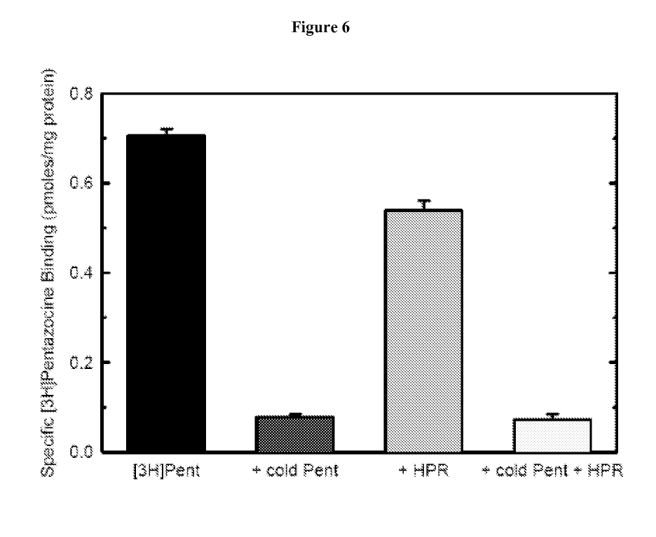

Two patient cohorts were selected. Each member of each patient cohort was a human that suffers from geographic atrophy (GA), secondary to age related macular degeneration (AMD). The mean concentration of serum retinol binding protein in the patients at baseline was approximately 60 μg/mL. The average concentration of serum retinol binding protein in normal individuals of the same type is about 25 μg/mL to about 40 μg/mL. In the first patient population, individuals were orally administered 100 mg/day fenretinide. In the second patient population, individuals were orally administered a placebo. The placebo group developed skin neoplasms (including basal cell carcinoma, squamous cell carcinoma, and/or melanoma) at a rate of 9.8%. In the first patient population, individuals developed cancer (including basal cell carcinoma, squamous cell carcinoma, and/or melanoma) at a rate of 2.5%. As illustrated in FIG. 3, individuals receiving 100 mg/day of a fenretinide agent had reduced incidences of cancer compared to those receiving placebo. FIG. 4 illustrates the cytotoxicity of fenretinide on the melanoma cell line (B16 (RL)). FIG. 5 illustrates the effect of fenretinide and compounds of Formula II (designated SIR-1001 and SIR-1005) on two human melanoma cell lines (M207 and SK-MEL28).

### Example 2

Patient cohorts are selected. Each member of each patient cohort is a human that suffers from elevated retinol binding protein (RBP) levels. Optionally, multiple cohorts are selected, e.g., with cohorts of patients having serum and/or tissue RBP levels of 20 μg/mL or less, 20 μg/mL to 30 μg/mL, 30 μg/mL to 40 μg/mL, 40 μg/mL to 50 μg/mL, 50 μg/mL to 60 μg/mL, and/or 60 μg/mL or above. Each cohort having a given RBP level is split into two sub-cohorts, a first receiving a dose of fenretinide agent (e.g., fenretinide or metabolite thereof) (e.g., orally at 100 mg/day), the second receiving a placebo. The rate of development of skin cancer (including basal cell carcinoma, squamous cell carcinoma, and/or melanoma) in the first group (receiving the fenretinide agent) and the second group (receiving placebo) are measured.

### Example 3

Patient cohorts are selected. Each member of each patient cohort is a human that suffers from elevated retinol levels. Optionally, multiple cohorts are selected, e.g., with cohorts of patients having serum and/or tissue retinol levels of 2 μmol/L or less, 2 μmol/L to 3 μmol/L, 3 μmol/L to 4 μmol/L, 4 μmol/L to 5 μmol/L, 5 μmol/L to 6 μmol/L, and/or 6 μmol/L or above. Each cohort having a given retinol level is split into two sub-cohorts, a first receiving a dose of fenretinide agent (e.g., fenretinide or metabolite thereof) (e.g., orally at 100 mg/day), the second receiving a placebo. The rate of development of skin cancer (including basal cell carcinoma, squamous cell carcinoma, and/or melanoma) in the first group (receiving the fenretinide agent) and the second group (receiving placebo) are measured.

### Example 4

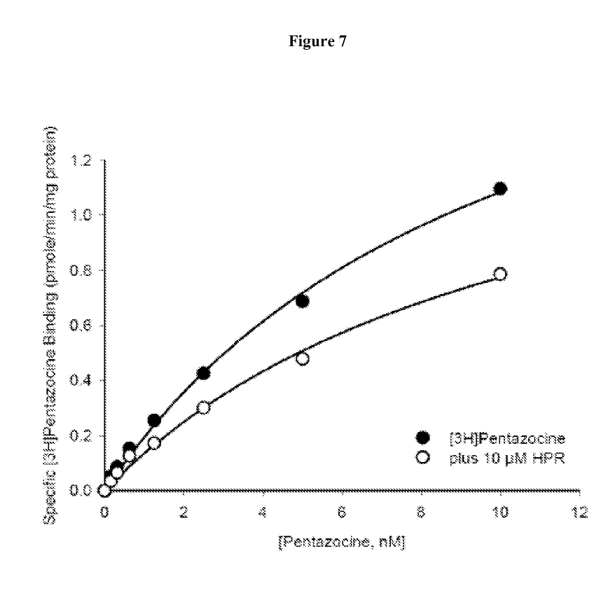

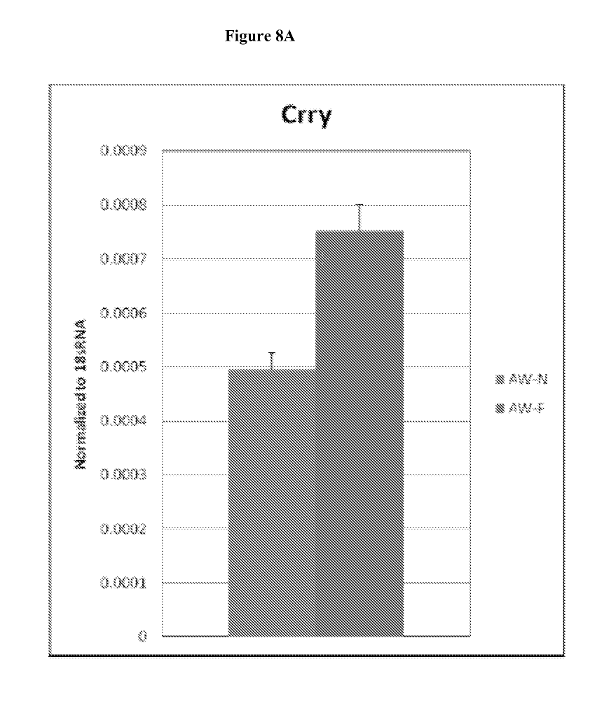

Sigma 1 receptor binding assays were performed according to DeHaven-Hudkins et al. (1992). In brief, guinea pig brain membrane aliquots were thawed, and then suspended at a concentration of 1 mg protein/ml by adding fresh sigma 1 assay buffer (50 mM Tris-HCl; pH 7.4, 25° C.). Each glass assay tube was kept at a final volume of 1.0 ml, contained 0.25 mg protein, and was incubated for 150 min at 37° C. with [3H](+)-pentazocine. [3H](+)-Pentazocine was used at either a single concentration (1.0 nM, FIG. 6) or at a range of concentrations (0-10 nM, FIG. 7). Nonspecific binding was defined by haloperidol (1.0 μM). Competing ligand concentration (e.g., fenretinide) was 10 μM. Assays were terminated by addition of ice-cold r1 assay buffer (5 ml) and filtration, using a cell harvester (Brandel, Gaithersburg, Md.), through glass fiber filters (GF/B) that had been pretreated with polyethyleneimine (0.5%) for 60 min. Tubes and filter discs were washed (3×3.5 ml) with ice-cold assay buffer, and the filter discs dried under vacuum. Scintillation counting was carried out after incubation of the discs with cocktail for at least 18 h.

FIGS. 6 and 7 illustrate that radio-labeled pentazocine ([3H]Pentazocine) has reduced binding when in the presence of fenretinide, indicating that fenretinide competes with pentazocine for binding to Sigma-1 receptor.

### Example 5

The effects of fenretinide on expression of complement genes was studies in BALC/C**mice.

Fenretinide supplemented chow (Ig fenretinide/kg chow), or control chow, was fed to pregnant mice. Birthed litters were maintained for 4 weeks. Mice were euthanized after 4 weeks and ocular tissue was prepared for RNA extraction. See FIG. 9 for results. Expression of complement genes was determined by RT-PCR. Data was normalized to expression of 18S RNA.

Litters of mice were separated into 2 groups just prior to weaning (4 weeks of age). Mice in Group I were fed fenretinide supplemented chow (1 g fenretinide/kg chow) for 7 weeks. Mice in Group II were fed control chow for 7 weeks. Mice were euthanized after 7 weeks and ocular tissue was prepared for RNA extraction. See FIG. 8 for results. Expression of complement genes was determined by RT-PCR. Data was normalized to expression of 18S RNA.

### Example 6

Patients with dry AMD were selected. Patients are divided into three groups. Group I (n=82) received a placebo. Group II (n=80) received fenretinide at a dose of 100 mg/day. Group III (n=84) received fenretinide at a dose of 300 mg/day. Incidence of all neoplasms and basal cell carcinoma was determined for each group. See Table 1 for results.

TABLE 1

| | Placebo | 100 mg fenretinide | 300 mg fenretinide |
|---|---|---|---|
| | (n = 82) | (n = 80) | (n = 84) |
| All neoplasms | 14 (17.1%) | 4 (5.0%) | 10 (11.9%) |
| Basal Cell | 5 (6.1%) | 2 (2.5%) | 3 (3.6%) |
| Carcinoma | | | |

1. A method of treating or reducing recurrence of a non-melanoma skin cancer in an individual diagnosed with excessive lipofuscin accumulation, a macular dystrophy, Stargardt's disease, GA, non-exudative AMD, and/or exudative AMD comprising administering to the individual an effective amount of an active agent that (a) decreases serum retinol; (b) increases ceramide levels; (c) decreases the activity of or blocks a sigma receptor; and/or (d) decreases the activity of or blocks the patched or smoothened receptor within the hedgehog pathway.

2. The method of claim 1, wherein the active agent is a retinoid or a retinoid derivative.

3. The method of claim 1, wherein the active agent is N-(4-hydroxyphenyl) retinamide, N-(4-methoxyphenyl)retinamide, 4-oxo-N-(4-hydroxyphenyl)retinamide, a compound of Formula (I): 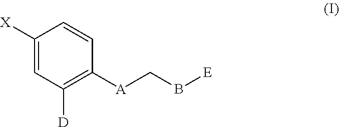
wherein:
A is O, NH, or S;
B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
D is isopropyl, isobutyl, sec-butyl, tert-butyl, neopentyl, sec-pentyl, isopentyl, cyclopropyl, cyclobutyl, cyclopentyl, methylenecyclopropyl, methylenecyclobutyl, methylenecyclopentyl;
E is (C═O)—OR, —O—(C═O)—R, —(C═O)—R, —OR, a carboxylic acid bioisostere, —(C═O)—NR¹R, NR¹—(C═O)—R, —(C₁-C₇)alkyl-(C═O)—OR, or —(C₁-C₇)alkyl-(C═O)—NR¹R;
R is H or 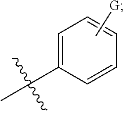
G is —OR¹, —(C₁-C₆)alkyl, —(C₁-C₆)alkyl-OR, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
X is a halogen;
or an active metabolite, or a pharmaceutically acceptable prodrug, salt, or solvate thereof;
or a combination thereof.

4. The method of claim 1, wherein the individual has elevated plasma retinol; elevated plasma RBP4; elevated concentrations of sigma receptors, optionally in an eye; or elevated concentrations of VEGF, optionally in an eye or a cancerous tumor; or a apo-RBP-to-holo-RBP ratio above 0.5.

5. The method of claim 1, wherein the individual is (a) a male human individual having plasma RBP4 concentration that is greater than 25 μg/mL and/or a plasma apo-RBP-to-holo-RBP ratio above 0.5; or (b) a female human individual having plasma RBP4 concentration that is greater than 20 μg/mL and/or a plasma apo-RBP-to-holo-RBP ratio above 0.5.

6. The method of claim 1, wherein the effective amount of the active agent is an amount sufficient to reduce the level of a risk factor associated with AMD or a non-melanoma skin cancer in the individual by about 25% to about 75%; wherein the risk factor is selected from: elevated concentrations of circulating vitamin A, elevated concentrations of circulating RBP, elevated concentrations of circulating holo-RBP, elevated concentrations of VEGF, or elevated concentrations of sigma receptors.

7. The method of claim 1, wherein the effective amount of the active agent is less than about 300 mg daily.

8. The method of claim 1, wherein the effective amount of the active agent is about 50 mg to about 150 mg daily.

9. The method of claim 1, wherein the skin cancer is a non-melanoma skin cancer.

10. The method of claim 1, wherein the skin cancer is basal cell carcinoma or squamous cell carcinoma.

11. Use of a retinoid or a retinoid derivative for the manufacture of a medicament for the treatment of non-melanoma skin cancer in an individual diagnosed with excessive lipofuscin accumulation, a macular dystrophy, Stargardt's disease, GA, non-exudative AMD, and/or exudative AMD.

12. The use of claim 11, wherein the retinoid or retinoid derivative is N-(4-hydroxyphenyl) retinamide, N-(4-methoxyphenyl)retinamide, 4-oxo-N-(4-hydroxyphenyl)retinamide, a compound of Formula (I): 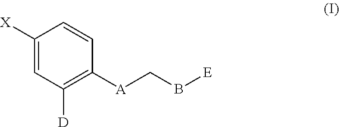
wherein:
A is O, NH, or S;
B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
D is isopropyl, isobutyl, sec-butyl, tert-butyl, neopentyl, sec-pentyl, isopentyl, cyclopropyl, cyclobutyl, cyclopentyl, methylenecyclopropyl, methylenecyclobutyl, methylenecyclopentyl;
E is (C═O)—OR, —O—(C═O)—R, —(C═O)—R, —OR, a carboxylic acid bioisostere, —(C═O)—NR¹R, NR¹—(C═O)—R, —(C₁-C₇)alkyl-(C═O)—OR, or —(C₁-C₇)alkyl-(C═O)—NR¹R;
R is H or 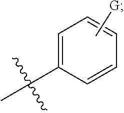
G is —OR¹, —(C₁-C₆)alkyl, —(C₁-C₆)alkyl-OR¹, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
X is a halogen;
or an active metabolite, or a pharmaceutically acceptable prodrug, salt, or solvate thereof;
or a combination thereof.

13. A method of treating Gorlin's Syndrome, comprising administering to the individual an effective amount of an active agent that (a) decreases serum retinol; (b) increases ceramide levels; (c) decreases the activity of or blocks a sigma receptor; and/or (d) decreases the activity of or blocks the patched or smoothened receptor within the hedgehog pathway.

14. The method of claim 13, wherein the active agent is a retinoid or a retinoid derivative.

15. The method of claim 13, wherein the active agent is N-(4-hydroxyphenyl) retinamide, N-(4-methoxyphenyl)retinamide, 4-oxo-N-(4-hydroxyphenyl)retinamide, a compound of Formula (I): 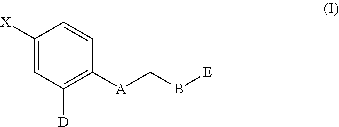
wherein:
A is O, NH, or S;
B is a bond, —(C₂-C₇)alkyl, —(C₂-C₇)alkenyl, —(C₃-C₈)cycloalkyl, —(C₂-C₇)heteroalkyl, —(C₃-C₈)heterocycloalkyl, —(C₃-C₈)cycloalkenyl, —(C₃-C₈)heterocycloalkenyl;
D is isopropyl, isobutyl, sec-butyl, tert-butyl, neopentyl, sec-pentyl, isopentyl, cyclopropyl, cyclobutyl, cyclopentyl, methylenecyclopropyl, methylenecyclobutyl, methylenecyclopentyl;
E is (C═O)—OR, —O—(C═O)—R, —(C═O)—R, —OR, a carboxylic acid bioisostere, —(C═O)—NR¹R, NR¹—(C═O)—R, —(C₁-C₇)alkyl-(C═O)—OR, or —(C₁-C₇)alkyl-(C═O)—NR¹R;
R is H or 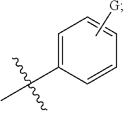
G is —OR¹, —(C₁-C₆)alkyl, —(C₁-C₆)alkyl-OR, halogen, —CO₂R¹, —(C₁-C₆)alkyl-CO₂R¹, NHR¹, —(C₁-C₆)alkyl-NHR¹, —(C═O)NHR¹, —(C₁-C₆)alkyl-(C═O)NHR¹, —NHR¹(C═O)R¹, —(C₁-C₆)alkyl-NHR¹(C═O)R¹;
R¹ is H or (C₁-C₆)alkyl;
X is a halogen;
or an active metabolite, or a pharmaceutically acceptable prodrug, salt, or solvate thereof;
or a combination thereof.

16. The method of claim 13, further comprising treating or reducing the recurrence of a basal cell carcinoma.

17. (canceled)

18. (canceled)
